(19) United States
(12) Patent Application Publication
(10) Pub. No.: US 2018/0036394 A1
(43) Pub. Date: Feb. 8, 2018

(54) PHARMACEUTICAL FORMULATIONS OF C1 ESTERASE INHIBITOR

(71) Applicant: CSL BEHRING GMBH, Marburg (DE)

(72) Inventors: Wolfram SCHAEFER, Muenchhausen (DE); Ernst-Juergen KANZY, Niederweimar (DE); Hubert METZNER, Marburg (DE); Frauke MAY, Marburg (DE); Ingo PRAGST, Edertal (DE)

(73) Assignee: CSL BEHRING GMBH, Marburg (DE)

(21) Appl. No.: 15/551,256

(22) PCT Filed: Feb. 19, 2016
(86) PCT No.: PCT/EP2016/053559

§ 371 Date: Aug. 15, 2017

(30) Foreign Application Priority Data
Feb. 20, 2015 (EP) 15156010.9

(51) Int. Cl. A61K 38/55; A61K 9/00

(52) U.S. Cl. CPC A61K 38/55; A61K 9/0019

(57) ABSTRACT

The present invention relates to pharmaceutical formulations comprising the C1 esterase inhibitor (“C1-INH”), exhibiting a higher stability for prolonged storage and a reduced kinematic viscosity for ameliorated use in treating or preventing disorders related to kinin formation.

### FIELD OF INVENTION

The present invention relates to pharmaceutical formulations comprising the C1 esterase inhibitor (“C1-INH”), exhibiting a higher stability for prolonged storage and a reduced kinematic viscosity for ameliorated use in treating or preventing disorders related to kinin formation.

### BACKGROUND

C1-INH, a plasma glycoprotein with a molecular weight of 104 kDa, belongs to the protein family of serine protease inhibitors (serpins), which regulate the activity of serine proteases by inhibiting their catalytic activity (Bock S C, et al., Biochemistry 1986, 25: 4292-4301). C1-INH inhibits the classical pathway of the complement system by inhibiting the activated serine proteases C1s and C1r. Furthermore, C1-INH is a major inhibitor of the contact activation system due to its ability to inhibit the activated serine proteases factor XIIa (FXIIa), factor XIa (FXIa), and plasma kallikrein (Davis A E, Clin. Immunol. 2005, 114: 3-9; Caliezi C et al., Pharmacol. Rev. 2000, 52: 91-112). Deficiency in C1-INH leads to the clinical manifestation of hereditary angioedema (HAE), which is characterized by episodes of acute angioedema attacks in subcutaneous or submucosal tissues such as the skin, larynx, or visceral organs (Longhurst H, et al. Lancet 2012, 379: 474-481) which last between 1 and 7 days and occur at irregular intervals. Abnormalities in C1-INH plasma content or in its functional activity (often referred to as a deficiency of functional C1-INH) result from various large and small mutations in the C1-INH gene (vide supra) (Karnaukhova E, J. Hematol. Thromb. Dis., 2013, 1-7).

Two types of hereditary C1-INH deficiency generally exist. The more prevalent type I HAE is characterized by low content (below 35% of normal) and low inhibitory activity of C1-INH in the circulation. Type II HAE is associated with normal or elevated antigenic levels of C1-INH of low functional activity. Recently, HAE with normal C1-INH (also known as type III HAE) has been described in two subcategories: (1) HAE due to mutation in the factor XII gene and, as a result, increased activity of factor XII leading to a high generation of bradykinin, and (2) HAE of unknown genetic cause. HAE attacks can be treated effectively by administering C1-INH (Longhurst H, et al., Lancet 2012, 379: 474-481; Bork K, Allergy Asthma Clin. Immunol. 2010, 6: 15). Moreover, administration of C1-INH has been shown to prevent edema formation in patients when given prophylactically. C1-INH is currently marketed e.g. as Berinert® (CSL Behring), Cetor® (Sanquin), Cinryze® (Shire), Ruconest®/Rhucin® (recombinant C1 inhibitor by Pharming). Due to its inhibitory effects on the complement and the contact activation systems, C1-INH substitution restores normal homeostatic function and inhibits the excessive formation of vasoactive peptides such as bradykinin, which mediate the formation of angioedema.

C1-INH has been reported to reduce ischemia-reperfusion injury in rodent models for cerebral ischemia-reperfusion (De Simoni et al., J Cereb Blood Flow Metab. 2003, 23: 232-9; Akita et al., 2003, Neurosurgery 52: 395-400).

The C1-INH compositions commercially available for the treatment of C1-INH deficiency up to date are all large volume formulations, i.e., these formulations need to be administered by intravenous injection. In view of the fact that C1-INH has been shown to prevent edema formation in patients with hereditary angioedema when given prophylactically (Cicardi M et al., Expert Opin. Pharmacother. 2007; 8: 3173-3181), there is a requirement for formulations that can be easily self-administered by the affected patients at regular intervals.

Long-term prophylaxis of HAE aims to prevent or to minimize the number and severity of angioedema attacks. However, the medications currently available for long-term prophylaxis are in many cases not optimal. Oral antifibrinolytics requiring multiple daily doses are relatively ineffective and frequently associated with significant side effects. Anabolic androgens are convenient to take and usually effective at doses <200 mg/day but can be associated with significant risk of serious side effects. Currently available formulations of C1-INH require intravenous access, imposing a burden on the patient, the healthcare provider, or both. Maintenance of intravenous access has required many patients to have venous ports implanted, which are associated with increased risks of infection and thrombosis. Plasma levels of functional C1-INH fall rapidly following intravenous administration of therapeutic dosages of C1-INH concentrates, reaching near basal levels within 3 days.

When manufacturing protein therapeutics, such as the C1-INH, regulatory authorities strongly recommend manufacturers of therapeutic protein products to minimize protein multimerisation and aggregation as much as possible. Moreover, an increased stability of protein therapeutics is highly desired for prolonged storage of such therapeutics. Conditions, which increase the protein's stability are also the best conditions to prevent denaturation and formation of high molecular weight components (HMWC) by multimerisation and aggregation, in particular of the therapeutic protein.

Therefore, strategies that minimize HMWC formation are highly desired to be developed as early as feasible in product development. This can be achieved by e.g. using an appropriate cell substrate, selecting manufacturing conditions that minimize HMWC formation, employing a purification scheme that removes HMWC to the greatest extent possible, choosing a container system, which minimizes HMWC formation of the protein, and most notably, choosing a formulation that minimizes HMWC formation, degradation and denaturation during storage.

Hence, formulation components are principally chosen based on their ability to preserve the native conformation of the therapeutic protein by preventing denaturation due to hydrophobic interactions that may lead to HMWC formation, as well as by preventing chemical degradation, including truncation, oxidation, and deamidation (Cleland et al., Crit. Rev. Ther. Drug Carrier Syst. 1993, 10(4): 307-377; Shire et al., J. Pharm. Sci. 2004, 93(6): 1390-1402; Wakankar and Borchardt, J. Pharm. Sci. 2006, 95(11): 2321-2336).

The potential clinical consequences of immune responses induced by protein HMWC may depend on the loss or preservation of native epitopes in the HMWC: (a) some antibodies generated by the human subject against HMWC containing native protein may bind to monomeric protein as well as to the HMWC and may inhibit or neutralize product activity. (b), other antibodies to denatured/degraded and hence aggregated protein bind uniquely to the HMWC material, but not to native protein monomers (Guidance for Industry Immunogenicity Assessment for Therapeutic Protein Products, U.S. Department of Health and Human Services, Food and Drug Administration, Center for Drug Evaluation and Research (CDER), Center for Biologics Evaluation and Research (CBER), August 2014).

The development of a concentrate comprising C1-INH (333 IU/mL) and hyaluronidase (rHuPH20) by Viropharma (now: Shire) in subcutaneous administration was stopped during clinical development: in particular almost all participants of the clinical trial suffered from adverse events at the injection site.

WO 2014/145519 discloses C1-INH compositions having about 400 or 500 U/mL C1-INH. It is suggested not to use citrate or citric acid as a buffer substance for subcutaneous administration. The disclosed formulations contain only particular buffer substances in low concentrations with no other excipient added. All disclosed C1-INH formulations have a relatively low overall purity of about 67% monomer content at t₀. Although the initial viscosity levels are within the limits usually set for use of an injectable product, there is a need for products with an even better viscosity. With regard to stability the WO 2014/145519 discloses only data after one week at 40° C. and after two weeks at 25° C., i.e. no long-term stability data are shown and thus long-term stability is unproven. In the present invention it has been found, that the long-term stability of the C1-INH formulations which are disclosed in WO 2014/145519 can be considerably improved.

In summary, there is a need for a C1-INH formulation that has a proven long-term storage stability by being less prone to HMWC formation as well as to denaturation and degradation, does not cause serious drug-related adverse events, cause only acceptable treatment-emergent adverse events at the injection site and has a low viscosity. Furthermore, a formulation, which is easily administered in high concentrations at low volumes, is urgently needed. In addition, it would be desirable that such a formulation can be used for prophylactic therapy as well as for acute therapy of patients suffering from hereditary angioedema.

### SUMMARY

The present invention provides low volume formulations comprising high concentrations of C1-INH and having an increased C1-INH stability, which makes said formulations well-suited for prolonged storage. Moreover, said formulations exhibit a reduced viscosity, which simplifies the subcutaneous and intravenous application of such compounds, in particular, as said formulations may be applied by the patients themselves.

The present invention further relates to use of such formulations in the acute and/or prophylactic treatment of disorders related to kinin formation.

In particular, the present invention relates to stable pharmaceutical formulations comprising
(a) C1-INH at a concentration of about 400 IU/mL-2,000 IU/mL; and
(b) sodium citrate having a calculated osmolarity of 20-120 mOsm/L or sodium di-hydrogen phosphate/di-sodium hydrogen phosphate having a calculated osmolarity of 60-120 mOsm/L; and
(c) one or more physiologically acceptable salt(s), other than the substances in (b), having a calculated osmolarity of 150-600 mOsm/L; or
   one or more amino acid(s) selected from glycine and/or one or more basic and/or one or more acidic L-amino acid(s) or a salt/salts thereof having a calculated osmolarity of 50-500 mOsm/L; or
   one or more physiologically acceptable salt(s), other than the substances in (b), and one or more amino acid(s) selected from glycine and/or one or more basic and/or one or more acidic L-amino acid(s) or a salt/salts thereof having together a calculated osmolarity of 80-740 mOsm/L;
wherein the overall calculated osmolarity of the formulation is 170-800 mOsm/L.

In various embodiments, the physiologically acceptable salt of the above formulation is a physiologically acceptable sodium salt, preferably selected from sodium chloride, disodium EDTA, sodium acetate, sodium succinate and sodium sulphate.

In various embodiments, the basic L-amino acid is arginine, lysine and/or histidine or a salt/salts thereof, preferably hydrochloride(s).

In various embodiments, the acidic L-amino acid is L-glutamic acid and/or L-aspartic acid or a salt/salts thereof, preferably sodium salt(s).

In various embodiments the pH of the pharmaceutical formulations referred to above is between about 6.7 and about 7.5.

In some embodiments, the pharmaceutical formulation comprises
(a) about 400-625 IU/mL C1-INH;
(b) about 20-120 mOsm/L sodium citrate;
(c) about 50-300 mOsm/L glycine; and
(d) about 190-400 mOsm/L sodium chloride,
wherein the overall calculated osmolarity of the formulation is 260-600 mOsm/L.

In various embodiments, the melting temperature of C1-INH measured by DSF in the above formulations is about 55° C. or higher, preferably about 55-60° C.

In various embodiments, the formulations above may further comprise
(a) a detergent selected from the group consisting of PS80 (polysorbate 80) and PS20 (polysorbate 20); and/or
(b) a preservative and/or antioxidant selected from the group consisting of benzylalcohol, cresol, phenol, methionine and glutathione.

In various embodiments, the C1-INH is human C1-INH. In preferred embodiments, the human C1-INH is derived from human plasma.

In various embodiments, the formulations referred to above comprise an absolute amount of C1-INH of at least 1,200 IU, at least 1,500 IU or at least 1,800 IU per finished dosage form.

In various embodiments, the above formulations are
(a) obtainable by reconstitution of a lyophilized powder with a suitable liquid, or
(b) provided as a liquid formulation.

In all of the above referenced embodiments, the formulation can be administered via subcutaneous administration or via intravenous administration, whereby optionally said formulation may be self-administered by the patient.

In various embodiments, the kinematic viscosity of the formulation is below 10 mm²/s, below 8 mm²/s, below 6 mm²/s or below 5 mm²/s.

In various embodiments, the formulation according to the invention comprises less than 10% of high molecular weight components (HMWC), less than 8% of HMWC, less than 5% of HMWC or less than 3% of HMWC.

Another aspect of the invention refers to pharmaceutical formulations for use
in the acute and/or prophylactic treatment of a disorder related to kinin formation, in particular hereditary angioedema (HAE), preferably HAE type I, HAE type II or HAE type III, secondary brain edema, edema of the central nervous system, hypotensive shock, or edema during or after contacting blood with an artificial surface;
in the acute and/or prophylactic treatment of a disorder related to an ischemia-reperfusion injury (IRI), in particular wherein the IRI is due to surgical intervention, in particular vascular surgery, cardiac surgery, neurosurgery, trauma surgery, cancer surgery, orthopedic surgery, transplantation, minimally invasive surgery, or insertion of a device for delivery of a pharmacologically active substance or for mechanical removal of complete or partial obstructions;
in the acute and/or prophylactic treatment of retinopathy; or
in preventing rejection of transplanted tissue in a patient.

Another aspect of the invention refers to kits comprising the pharmaceutical formulation of the invention as a lyophilized powder and a respective volume of a suitable liquid for reconstitution. Yet another aspect of the invention refers to kits comprising the pharmaceutical formulation of the invention and at least one syringe and/or one needle. And yet another aspect refers to a syringe prefilled with a liquid pharmaceutical formulation of the invention.

### DETAILED DESCRIPTION

### Definitions

According to the present invention, the term “C1 esterase inhibitor” or “C1 inhibitor” (“C1-INH”) refers to the proteins or fragments thereof that function as serine protease inhibitors and inhibit proteases associated with the complement system, preferably proteases C1r and C1s as well as MASP-1 and MASP-2, with the kallikrein-kinin system, preferably plasma kallikrein and factor XIIa, and with the coagulation system, preferably factor XIa and factor XIIa. In addition, the C1-INH can serve as an anti-inflammatory molecule that reduces the selectin-mediated leukocyte adhesion to endothelial cells. C1-INH as used herein can be the native serine protease inhibitor or an active fragment thereof, or it can comprise a recombinant peptide, a synthetic peptide, peptide mimetic, or peptide fragment that provides similar functional properties, such as the inhibition of proteases C1r and C1s, and/or MASP-1 and MASP-2, and/or plasma kallikrein, and/or factor XIIa, and/or factor XIa. The term C1-INH shall also encompass all natural occurring alleles, splice variants and isoforms which have the same or similar functions as the C1-INH. For further disclosure regarding the structure and function of C1-INH, see U.S. Pat. No. 4,915,945, U.S. Pat. No. 5,939,389, U.S. Pat. No. 6,248,365, U.S. Pat. No. 7,053,176 and WO 2007/073186.

One “unit” (“U”) of C1-INH is equivalent to the C1-INH activity in 1 mL of fresh citrated plasma of healthy donors. The C1-INH may also be determined in “international units” (“IU”). These units are based on the current World Health Organization (WHO) standard for C1-INH concentrates (08/256) which was calibrated in an international collaborative study using normal local human plasma pools. In general, U and IU are equivalent.

The term “hereditary angioedema” (“HAE”) as used herein relates to angioedema caused by a low content and low inhibitory activity of C1-INH in the circulation (HAE type I) or by the presence of normal or elevated antigenic levels of C1-INH of low functional activity (HAE type II). The term “HAE” as used herein also encompasses HAE with normal C1-INH (also known as HAE type III) which has been described recently in two subcategories: (1) HAE due to mutation in the factor XII gene and, as a result, increased activity of factor XII leading to a high generation of bradykinin, and (2) HAE of unknown genetic cause. In patients suffering from hereditary angioedema, edema attacks can occur in various intervals, including a daily, weekly, monthly, or even yearly basis. Furthermore, there are affected patients wherein no edema occurs.

The term “angioedema” (“edema”) as used herein relates to swelling of tissue, for example swelling of skin or mucosa. The swelling can occur, for example, in the face, at hands or feet or on the genitals. Furthermore, swelling can occur in the gastro-intestinal tract or in the respiratory tract. Other organs can also be affected. Swelling persists usually between one and three days. However, remission can already occur after hours or not until weeks.

The term “ischemia-reperfusion injury” (“IRI”) is an injury caused by the return of blood into tissue (“reperfusion”) after an ischemia or a lack of oxygen. Direct damage to the tissue is caused by the interruption of the blood flow, mainly due to loss of oxygenation to the viable tissue, ultimately leading to infarction if not reversed. However, if the insult is reversed, the reperfusion of the ischemic tissue may paradoxically cause further “indirect” damage. Upon long duration of ischemia, the “direct” damage resulting from hypoxia alone is the predominant mechanism. For shorter durations of ischemia, the “indirect” reperfusion mediated damage increasingly contributes to the damage caused.

The term “retinopathy” as used herein relates to acute or persistent damage of the eye. Retinopathy can be caused by diabetes mellitus (leading to diabetic retinopathy), arterial hypertension (leading to hypertensive retinopathy), prematurity of the newborn (leading to retinopathy of prematurity), exposure to ionizing radiation (radiation retinopathy), direct sunlight exposure (solar retinopathy), sickle cell disease, retinal vascular disease such as retinal vein or artery occlusion, trauma, especially to the head and other diseases or conditions. Many types of retinopathy are proliferative resulting, most often, from neovascularization or the overgrowth of blood vessels. Angiogenesis, the sprouting of new vessels is the hallmark precursor that may result in blindness or severe vision loss particularly if the macula becomes affected. In rare cases, retinopathy is caused by genetic diseases.

The term “acute treatment” or “treatment” as used herein relates to the treatment of a patient displaying acute symptoms. Acute treatment can occur from the appearance of the symptom until the full remission of the symptom. An acute treatment can occur once or several times until the desired therapeutic effect is achieved.

The term “prophylactic treatment” or “prophylaxis” or “prevention” as used herein relates to the treatment of a patient in order to prevent the occurrence of symptoms. Prophylactic treatment can occur at regular intervals of days, weeks or months. Prophylactic treatment can also occasionally occur.

The term “about” means within an acceptable error range for a particular value which partially depends on the limitations of the measurement system.

The term “HMWC” or “high molecular weight components” as used herein refers to any self-associated, i.e. multimerised or aggregated protein species, in particular of C1-INH, with monomer defined as the smallest functional subunit. HMWC are further classified based on five characteristics: size, reversibility/dissociation, conformation, chemical modification, and morphology (Narhi et al., J. Pharm. Sci. 2012, 101(2): 493-498).

HMWC, in particular multimers and aggregates, have been recognized for their potential to elicit immune responses to therapeutic protein products for over a half-century (Gamble, Int. Arch. Allergy Appl. Immunol. 1966, 30(5): 446-455). The underlying mechanisms by which protein HMWC may elicit or enhance immune responses include inter alia the following: extensive cross-linking of B-cell receptors, causing efficient B-cell activation (Dintzis et al., J. Immunol. 1989, 143(4): 1239-1244; Bachmann et al., Science 1993, 262(5138): 1448-1451); enhancing antigen uptake, processing, and presentation; and triggering immunostimulatory danger signals (Seong and Matzinger, Nat. Rev. Immunol. 2004, 4(6): 469-478). Such mechanisms may enhance recruitment of T-cell help needed for generation of high-affinity, isotype-switched IgG antibody, whereby the antibody response is most often associated with neutralization of product efficacy (Bachmann and Zinkernagel, Annu. Rev. Immunol. 1997, 15:235-70).

The term “finished dosage form (FDF)” of a drug is a dosage form of the drug which has undergone all stages of manufacture, including packaging in its final container and labelling.

The term “physiologically acceptable salt” of this invention refers to salts in formulations that are mainly used in treating medical conditions in humans, in particular to treating or preventing disorders related to kinin formation. Further, a physiological acceptable salt refers to ionic substances which are soluble, i.e. in the liquid, preferably aqueous, state a physiological acceptable salt will be present in form of its dissolved cation(s) and anion(s), and which will not cause serious adverse side events after administration to the human body. In this sense, the formulations or their finished dosage forms are appropriate for physiological practice together with other excipients.

The term “osmotic concentration”, or “osmolarity”, is the measure of solute concentration, defined as the number of osmoles (Osm) of solutes per litre (L) of solution (osmol/L or Osm/L). Osmolarity measures the number of osmoles of solute particles per unit volume of solution, whereas molarity measures the number of moles of solutes per unit volume of solution.

Osmolarity can be measured e.g. by measuring the freezing point depression by methods known to the person skilled in the art. Methods for measuring the osmolarity of a solution by freezing point depression are described, for example, in the European Pharmacopeia 2.2.35 and the U.S. Pharmacopeia chapter 785. For example, for water, 1 Osmol of a solute added to 1 kg of water lowers the freezing point by 1.86° C.

The theoretical calculation of osmolarity is well known to the person skilled in the art. Briefly, one calculates for each component of the solution the product of the osmotic coefficient f, the number of particles n into which the molecule dissociates in water, and the molar concentration of that component, and sums the result over all components. Hence, the osmolarity (in Osm/L) of a solution can be calculated as follows:

Osm/L=ΣfᵢnᵢCᵢ
index i represents the identity of a particular component, such as a salt ion;
f is the osmotic coefficient for that particular component;
n is the number of particles into which the molecule of that particular component dissociates in water;
C is the molar concentration of that particular component.

The molar concentration has slight temperature dependence; for the present purpose it is referred to the concentrations at 2-35° C., preferably 10-30° C., more preferably 25° C. at atmospheric pressure.

At concentrations typically used in the claimed compositions for injection assuming an osmotic coefficient of 1 for each particular component gives a sufficient approximation for calculating the osmolarity of a solution. For example, sodium chloride is a particular component which dissociates in water into two particles; the osmolarity of sodium chloride is twofold of its molar concentration. Sodium di-hydrogen phosphate/di-sodium hydrogen phosphate is in the applicable pH range approximately an equimolar mixture of both components, so that it will dissociate in water into two or three particles, respectively; the osmolarity of phosphate is 2.5-fold of its molar concentration. As used herein the molar concentrations of C1-NH in the claimed formulations are very low and can be disregarded while calculating the osmolarity. It is well known that deviations between the measured osmolarity and the calculated osmolarity may occur. In particular in solutions where highly charged proteins, such as C1-INH, are present there might be a strong influence of that protein to the measured osmolarity due to the Donan potential. Hence, the term “osmolarity” or “calculated osmolarity” of this invention refers to the calculated osmolarity.

The term “DSF” refers to “differential scanning fluorimetry”, which is a thermal shift assay or thermal denaturation assay that measures the stability of a target protein and a subsequent increase in the melting temperature of a protein upon binding of a ligand to the protein. The binding of low molecular weight ligands can increase the thermal stability of a protein. This stability change is measured by performing a thermal denaturation curve in the presence of a fluorescent dye, such as Sypro Orange. When the protein unfolds, the exposed hydrophobic surfaces bind the dye, resulting in an increase in fluorescence. The stability curve and its midpoint value (melting temperature mt) are obtained by gradually increasing the temperature to unfold the protein and measuring the fluorescence at each point. In other words, the DSF monitors thermal unfolding of proteins in the presence of a fluorescent dye and the temperature at which a protein unfolds is measured by an increase in the fluorescence of that dye with affinity for hydrophobic parts of protein, which are exposed as the protein unfolds. The difference in melting temperature can be used to rank buffer conditions, additives, according to their enhancement of protein stability.

The melting temperature (mt) is defined by the Gibbs free energy of unfolding (ΔGu). An increase of temperature leads to a decrease of the portion of folded protein, and a decrease of ΔGu. ΔGu equals zero at a state, where the ratio of folded and unfolded proteins is equal. The corresponding temperature is the melting temperature of a protein. Hence, the melting temperature is a measure for assessing the thermal stability of a protein. The formulations of this invention offer an increase of melting temperatures as compared to prior art C1-INH formulations, as can be seen in Tables 2-3. A higher stability of such formulations makes them more suited for longer C1-INH storage.

The term “WFI” refers to “water for injection”. It is water intended for use in the manufacture of medicines for parenteral administration, the solvent of which is water. Alternatively, it refers to water that is used to dissolve or dilute substances or preparations for parenteral administration. It is purified by distillation or a purification process, which is equivalent or superior to distillation in the removal of chemicals and microorganisms.

The kinematic viscosity (m²/s) is the ratio between the dynamic viscosity [Pa*s=kg/m·s] and the density of a fluid [kg/m³].

The terms “sodium citrate” or “Na-citrate” or “Na₃-citrate” of this invention refers to Tri-sodium-citrate, i.e. Na₃C(OH)(COO⁻)(CH₂COO⁻)₂.

### C1 Esterase Inhibitor

In certain embodiments of the invention, the C1-INH is a plasma-derived or a recombinant C1-INH. In further embodiments said inhibitor is identical to the naturally occurring human protein or a variant thereof. In other embodiments, said inhibitor is human C1-INH. In other embodiments, said inhibitor is a recombinant analogue of human C1-INH protein.

According to the present invention, the C1-INH may be modified to improve its bioavailability and/or half-life, to improve its efficacy and/or to reduce its potential side effects. The modification can be introduced during recombinant synthesis or otherwise. Examples for such modifications are glycosylation, PEGylation and HESylation of the C1-INH or an albumin fusion of the described C1-INH. In some embodiments, C1-INH comprises a fusion construct between C1-INH and albumin, in particular human albumin. In some embodiments, the albumin is a recombinant protein. In certain embodiments, the C1-INH and albumin proteins are joined directly, or via a linker polypeptide. For further disclosure regarding glycosylation and albumin fusion of proteins, see WO 01/79271.

### Preparation of C1-INH

For the purpose of this invention, the C1-INH can be produced according to methods known to the skilled person. For example, plasma-derived C1-INH can be prepared by collecting blood plasma from several donors. Donors of plasma should be healthy as defined in the art. Preferably, the plasma of several (1000 or more) healthy donors is pooled and optionally further processed. An exemplary process for preparing C1-INH for therapeutic purposes is disclosed in U.S. Pat. No. 4,915,945. Alternatively, in other embodiments, C1-INH can be collected and concentrated from natural tissue sources using techniques known in the art. Recombinant C1-INH can be prepared by known methods.

In certain embodiments, C1-INH is derived from human plasma. In further embodiments, C1-INH is prepared by recombinant expression.

A commercially available product comprising C1-INH is, e.g., plasma-derived Berinert® (CSL Behring). Berinert® is manufactured according to A. Feussner et al. (Transfusion 2014, 54: 2566-73) and is indicated for treatment of hereditary angioedema and congenital deficiencies. Alternative commercially available products comprising C1-INH are plasma-derived Cetor® (Sanquin), Cinryze® (Shire), and recombinant Ruconest®/Rhucin® (Pharming).

### C1-INH Formulations

The present invention relates to stable pharmaceutical formulations comprising (a) C1-INH. These highly concentrated formulations of the invention are provided in low volume formulations having a low viscosity. The formulations are well-tolerated and suitable for intravenous and in particular subcutaneous administration.

The concentration of C1-INH in said formulations is about 400 IU/mL to 2,000 IU/mL, preferably of about 400 IU/mL to 1,200 IU/mL, more preferably of about 400 IU/mL to 1000 IU/mL, more preferably of about 400 IU/mL to 800 IU/mL, more preferably of about 400 IU/mL to 650 IU/mL, and most preferably of about 500 IU/mL or any range in between.

Said formulations further comprise:

(b) sodium citrate having a calculated osmolarity of 20-120 mOsm/L or sodium di-hydrogen phosphate/di-sodium hydrogen phosphate having a calculated osmolarity of 60-120 mOsm/L; and
(c) one or more physiologically acceptable salt(s), other than the substances in (b), having a calculated osmolarity of 150-600 mOsm/L; or
one or more amino acid(s) selected from glycine and/or one or more basic and/or acidic L-amino acid(s) or salts thereof having a calculated osmolarity of 50-500 mOsm/L; or
one or more physiologically acceptable salt(s), other than the substances in (b), and one or more amino acid(s) selected from glycine and/or one or more basic and/or acidic L-amino acid(s) or salts thereof having together a calculated osmolarity of 80-740 mOsm/L,
wherein the overall calculated osmolarity of the formulation is 170-800 mOsm/L.

In certain embodiments, the calculated osmolarity of sodium citrate is 80 to 120 mOsm/L, more preferably, more than 80 mOsm/L to 120 mOsm/L. Alternatively, in other certain embodiments, the calculated osmolarity of sodium citrate is 30 to 80 mOsm/L, more preferably 40 to 70 mOsm/L.

In other certain embodiments, the calculated osmolarity of sodium di-hydrogen phosphate/di-sodium hydrogen phosphate is 60-90 mOsm/L.

Further, in certain embodiments, the one or more physiologically acceptable salt(s), other than the substances in (b), have a calculated osmolarity of 170-500 mOsm/L, more preferably 230-470 mOsm/L. In other certain embodiments, the one or more amino acid(s) have a calculated osmolarity of 170-500 mOsm/L, more preferably 230-470 mOsm/L. Alternatively, the one or more physiologically acceptable salt(s), other than the substances in (b), and the one or more amino acid(s) have together a calculated osmolarity of 150-600 mOsm/L, more preferably 230-470 mOsm/L.

In other embodiments, the overall calculated osmolarity of the formulation is 230-700 mOsm/L, more preferably 250-600 mOsm/L. Even more preferably, the osmolarity is 270-330 mOsm/L, most preferably 280-330 mOsm/L or any range in between. In other, even more preferred embodiments, the osmolarity is 400-600 mOsm/L, most preferably 450-550 mOsm/L or any range in between.

In various embodiments, the physiologically acceptable salt is an alkali metal and/or alkaline earth metal salt. In certain embodiments, the physiologically acceptable salt is a sodium, potassium, magnesium or calcium salt, preferably a sodium salt.

In various embodiments, the base of the physiologically acceptable salt can be selected from, but not restricted to carbonate, sulphate, halide such as chloride, and carboxyl-group-comprising base such as EDTA, acetate, succinate, malate, maleates, and tartrates. In certain embodiments, the base of the physiologically acceptable salt is a chloride, sulphate, acetate or succinate.

In certain embodiments the physiologically acceptable salt(s) is/are selected from NaCl, Na₂SO₄, Na-Acetate and Na-Succinate.

In certain embodiments the one or more basic L-amino acid(s) is/are selected from L-arginine, L-histidine, L-lysine or salts thereof, preferably hydrochlorides. In some embodiments the one or more acidic L-amino acid(s) is/are selected from L-glutamic acid and L-aspartic acid or salts, preferably sodium salts, thereof.

In certain embodiments the pharmaceutical formulation comprises only one type of amino acid.

In certain embodiments the pharmaceutical formulation does not comprise a tissue permeability enhancer, such as e.g. hyaluronidase.

In various embodiments, the pH of the pharmaceutical formulation is between about 6.7 and 7.5, between about 6.8 and 7.4, between about 6.9 and 7.3, between about 7.0 and 7.2 or any range in between. In the most preferred embodiment, the pH of the formulation is about 7.0, about 7.1 or about 7.2.

In some embodiments, the pharmaceutical formulation comprises

(a) about 400-625 IU/mL C1-INH;
(b) about 20-120 mOsm/L sodium citrate;
(c) about 50-300 mOsm/L glycine; and
(d) about 190-400 mOsm/L sodium chloride,
wherein the overall calculated osmolarity of the formulation is about 260-800 mOsm/L.

In preferred embodiments, the pharmaceutical formulation comprises

(a) about 400-625 IU/mL C1-INH;
(b) about 40-60 mOsm/L sodium citrate;
(c) about 100-200 mOsm/L glycine; and
(d) about 240-350 mOsm/L sodium chloride,
wherein the overall calculated osmolarity of the formulation is about 380-600 mOsm/L.

In more preferred embodiments, the pharmaceutical formulation comprises

(a) about 450-550 IU/mL C1-INH;
(b) about 40-60 mOsm/L sodium citrate;
(c) about 100-200 mOsm/L glycine; and
(d) about 240-350 mOsm/L sodium chloride,
wherein the overall calculated osmolarity of the formulation is 380-600 mOsm/L.

In even more preferred embodiments, the pharmaceutical formulation comprises:

(a) about 490-510 IU/mL C1-INH;
(b) about 40-60 mOsm/L sodium citrate;
(c) about 100-200 mOsm/L glycine; and
(d) about 240-350 mOsm/L sodium chloride,
wherein the overall calculated osmolarity of the formulation is 380-600 mOsm/L.

In one embodiment, the above formulation comprises in addition to the C1-INH about 13 mM Na-Citrate, about 133 mM Glycine, and about 154 mM NaCl at a pH of 6.9-7.1.

In certain embodiments, the pharmaceutical formulation comprises

(a) about 400-625 IU/mL C1-INH;
(b) about 40-120 mOsm/L sodium citrate;
(c) about 200-600 mOsm/L sodium sulphate,
wherein the overall calculated osmolarity of the formulation is about 250-720 mOsm/L.

In preferred embodiments, the pharmaceutical formulation comprises

(a) about 400-625 IU/mL C1-INH;
(b) about 40-80 mOsm/L sodium citrate;
(c) about 200-500 mOsm/L sodium sulphate,
wherein the overall calculated osmolarity of the formulation is about 280-580 mOsm/L.

In some embodiments, the pharmaceutical formulation comprises

(a) about 400-625 IU/mL C1-INH;
(b) about 40-80 mOsm/L sodium citrate;
(c) about 200-310 mOsm/L sodium sulphate,
wherein the overall calculated osmolarity of the formulation is about 280-350 mOsm/L.

In other embodiments, the pharmaceutical formulation comprises

(a) about 400-625 IU/mL C1-INH;
(b) about 40-80 mOsm/L sodium citrate;
(c) about 400-500 mOsm/L sodium sulphate,
wherein the overall calculated osmolarity of the formulation is about 400-550 mOsm/L.

In particular embodiments the pharmaceutical formulation comprises the excipients with concentrations as disclosed in Table 1 at a pH of 7.0, 7.1 or 7.2.

In various embodiments, the melting temperature of C1-INH measured by DSF in the disclosed formulations is about 55° C. or higher, about 56° C. or higher, or about 57° C. or higher. In certain embodiments the melting temperature measured by DSF is about 55-60° C., about 56-60° C. or about 57-60° C. The melting temperature measured by DSF has a measurement margin of about +/−1° C.

In further embodiments, the provided formulations comprise one or more detergents and/or one or more preservatives and/or one or more antioxidants.

In certain embodiments, the pharmaceutical formulation can comprise PS80 (polysorbate 80) and/or PS20 (polysorbate 20). In the pharmaceutical formulation, PS80 and/or PS20 may be present at a concentration of about 0.5-2 mg/mL.

In certain embodiments, the preservatives and/or antioxidants are selected from the group consisting of benzylalcohol, cresol, phenol, methionine and glutathione. In the pharmaceutical formulation the preservatives and/or antioxidants may be present at a concentration of about 1-5 mM.

The provided formulations may comprise further pharmaceutical carriers and excipients that are well known in the art (see for example “Pharmaceutical Formulation Development of Peptides and Proteins”, Frokjaer et al., Taylor & Francis 2000 or “Handbook of Pharmaceutical Excipients”, 3^{rd} edition, Kibbe et al., Pharmaceutical Press 2000).

In some embodiments, the C1-INH is human C1-INH. In certain embodiments the human C1-INH is derived from human plasma, or the human C1-INH is recombinantly expressed. Preferably, the C1-INH is derived from human plasma.

In some embodiments, the formulation comprises an absolute amount of C1-INH of about 1,000 IU/FDF, 1,200 IU/FDF, about 1,500 IU/FDF, about 1,800 IU/FDF, about 2,100 IU/FDF, about 2,400 IU/FDF, about 2,700 IU/FDF or about 3,000 IU/FDF or any absolute amount in between. In preferred embodiments, the formulation comprises an absolute amount of C1-INH of at least 1,200 IU/FDF, 1,500 IU per FDF, or at least 1,800 IU per FDF.

In various embodiments, the liquid pharmaceutical formulation is provided in a volume of about 0.1-10 mL/FDF, about 1-5 mL/FDF or about 3 mL/FDF or any volume in between. In preferred embodiments, the formulation is provided as an aqueous solution in a volume of about 3 mL per FDF, of about 4 mL per FDF or of about 6 mL per FDF.

In various embodiments, the kinematic viscosity of the formulation as determined immediately after preparation of the formulation is below 10 mm²/s, preferably below 8 mm²/s, more preferably below 6 mm²/s or even more preferably below 5 mm²/s. Immediately after preparation means within one day after preparation. Determination of the kinematic viscosity is done at +20° C. Preferably the kinematic viscosity of the three samples is measured using a capillary viscometer according to Ubbelohde, e.g. by Schott-Geräte GmbH, Hofheim, Germany. To obtain the dynamic viscosity, the kinematic viscosity has to be multiplied with the mass density of the corresponding solutions which can be assumed to be about 1 g/cm³ (Dynamic Viscosity (cP)=Kinematic Viscosity (cSt)*Density (kg/m³)).

In various embodiments, the pharmaceutical formulations of the invention comprises less than 10% of HMWC, preferably less than 8% of HMWC, more preferably less than 5% of HMWC or even more preferably less than 3% of HMWC as determined immediately by SEC-HPLC. Immediately after preparation means within one day after preparation.

In certain various embodiments, the kinematic viscosity of the formulation determined as described above is below 10 mm²/s, preferably below 8 mm²/s, more preferably below 6 mm²/s and even more preferably below 5 mm²/s and the formulation comprises less than 10%, preferably less than 8%, more preferably less than 5%, and most preferably less than 3% HMWC.

In some embodiments, a pharmaceutical formulation is provided, whereby said formulation is
(a) obtainable by reconstitution of a lyophilized powder with a suitable liquid, or
(b) provided as a liquid formulation.

In other words, in various embodiments, the components of the pharmaceutical formulation are present in a solution suitable for injection without any further processing, i.e. the formulation is provided as a stable liquid formulation. Alternatively, the components are provided as a stable lyophilized powder and the indicated concentrations are reached upon reconstitution of the lyophilized powder in the respective volume of a suitable liquid. The liquid suitable for reconstitution comprises water for injection (WFI). Preferably the liquid suitable for reconstitution is WFI.

The terms “stable formulation”, “stable lyophilized powder”, “stable lyophilized formulation” or “stable liquid formulation” as used herein refer to pharmaceutical formulations wherein no significant decrease of C1-INH activity is observed after a certain period of time of storage at least at 2-8° C., preferably at about 25° C. The term “no significant decrease of C1-INH activity” means at least 70%, 75%, 80%, 85%, 90%, 95% 96%, 97%, 98%, 99% of the C1-INH activity of the original C1-INH activity. The term “C1-INH activity” refers to the inhibitory activity of the C1-INH protein in plasma and is indicated in “IU/mL” and can be measured without limitation e.g. by a chromogenic assay.

In alternative embodiments, the terms “stable formulation”, “stable lyophilized powder”, “stable lyophilized formulation” or “stable liquid formulation” as used herein refer to formulations wherein no significant increase of HMWC formation is observed after a certain period of time of storage at least at 2-8° C., preferably at about 25° C. The term “no significant increase of HMWC formation” means at most 20%, 15%, 14%, 13%, 12%, 11% 10%, 9%, 8%, 7%, 6%, 5% protein HMWC at a certain point of time, e.g. after 1, 2, 3, 4, 5, 6, 7, 8, 9, 10 11, 12, 15, 18, 21 or 24 months, i.e. the percentage of HMWC is determined as the percentage of HMWC of the total protein content in the formulation at this certain point of time and this value is at the most 20%, 15%, 14%, 13%, 12%, 11% 10%, 9%, 8%, 7%, 6%, 5%. The level of HMWC of the total protein content can be measured without limitation e.g. by SEC HPLC (see e.g. example 3).

In alternative embodiments, the terms “stable formulation”, “stable lyophilized powder”, “stable lyophilized formulation” or “stable liquid formulation” as used herein refer to formulations wherein no significant increase of fragmentation is observed after a certain period of time of storage at least at 2-8° C., preferably at about 25° C. The term “no significant increase of fragmentation” means at most 20%, 15%, 14%, 13%, 12%, 11% 10%, 9%, 8%, 7%, 6%, 5% fragments at a certain point of time, e.g. after 1, 2, 3, 4, 5, 6, 7, 8, 9, 10 11, 12, 15, 18, 21 or 24 months, i.e. the percentage of fragments is determined as the percentage of fragments of the total protein content in the formulation at this certain point of time and this value is at the most 20%, 15%, 14%, 13%, 12%, 11% 10%, 9%, 8%, 7%, 6%, 5%. The level of fragments of the total protein content can be measured without limitation e.g. by SEC HPLC (see e.g. example 3).

In alternative embodiments, the terms “stable formulation”, “stable lyophilized powder”, “stable lyophilized formulation” or “stable liquid formulation” as used herein refer to formulations wherein the melting temperature measured by DSF is about 55° C. or higher, about 56° C. or higher, or about 57° C. or higher. In further embodiments, the melting temperature of a stable formulation measured by DSF is about 55-60° C., about 56-60° C. or about 57-60° C. In general, the melting temperature measured by DSF has a measurement margin of about +/−1° C.

In a series of embodiments, the formulations provided herein upon lyophilization will be stable for a certain period of time, i.e. for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more months at a temperature of 25° C. In a preferred embodiment, the lyophilized formulation will be stable for at least 6 months (25° C.). In a more preferred embodiment, the lyophilized formulation will be stable for at least 12 months (25° C.). In another preferred embodiment, the lyophilized formulation will be stable for at least 24 months (25° C.).

In another series of embodiments, the liquid formulation provided herein will be stable for a certain period of time, i.e. for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more months at a temperature of 2-8° C. In a preferred embodiment, the liquid formulation will be stable for at least 6 months (2-8° C.). In a more preferred embodiment, the liquid formulation will be stable for at least 12 months (2-8° C.). In another preferred embodiment, the liquid formulation will be stable for at least 24 months (2-8° C.).

In another series of embodiments, the liquid formulation provided herein will be stable for a certain period of time, i.e. for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more months at a temperature of 25° C. In a preferred embodiment, the liquid formulation will be stable for at least 6 months (25° C.). In a more preferred embodiment, the liquid formulation will be stable for at least 12 months (25° C.).

In certain embodiments, the provided liquid formulations retain at least 70%, preferably at least 80%, more preferably at least 90% and most preferably at least 95% C1-INH activity after 1 month of storage at 2-8° C.

In certain embodiments, the provided liquid formulations retain at least 70%, preferably at least 80%, more preferably at least 90% and most preferably at least 95% C1-INH activity after 6 months of storage at 2-8° C.

In certain embodiments, the provided liquid formulations retain at least 70%, preferably at least 80%, more preferably at least 90% and most preferably at least 95% C1-INH activity after 12 months of storage at 2-8° C.

In certain embodiments, the provided liquid formulations retain at least 70%, preferably at least 80%, more preferably at least 90% and most preferably at least 95% C1-INH activity after 1 month of storage at 25° C.

In certain embodiments, the provided liquid formulations retain at least 70%, preferably at least 80%, more preferably at least 90% and most preferably at least 95% C1-INH activity after 6 months of storage at 25° C.

In certain embodiments, the provided liquid formulations retain at least 70%, preferably at least 80%, more preferably at least 90% and most preferably at least 95% C1-INH activity after 12 months of storage at 25° C.

In certain embodiments, the provided liquid formulations retain at least 30%, preferably at least 40%, more preferably at least 50% and most preferably at least 60% C1-INH activity after 1 month of storage at 35° C.

In certain embodiments, the provided liquid formulations retain at least 30%, preferably at least 40%, more preferably at least 50% and most preferably at least 60% C1-INH activity after 6 months of storage at 35° C.

In certain embodiments, the provided liquid formulations retain at least 30%, preferably at least 40%, more preferably at least 50% and most preferably at least 60% C1-INH activity after 12 months of storage at 35° C.

In certain embodiments, the provided lyophilized formulation retains at least 70%, preferably at least 80%, more preferably at least 90% and most preferably at least 95% C1-INH activity after 6 months of storage at 25° C.

In certain embodiments, the provided lyophilized formulation retains at least 70%, preferably at least 80%, more preferably at least 90% and most preferably at least 95% C1-INH activity after 12 months of storage at 25° C.

In certain embodiments, the provided lyophilized formulation retains at least 70%, preferably at least 80%, more preferably at least 90% and most preferably at least 95% C1-INH activity after 24 months of storage at 25° C.

In certain embodiments, the provided lyophilized formulation retains at least 70%, preferably at least 80%, more preferably at least 90% and most preferably at least 95% C1-INH activity after 48 months of storage at 25° C.

In certain embodiments, the provided lyophilized formulation retains at least 30%, preferably at least 40%, more preferably at least 50% and most preferably at least 60% C1-INH activity after 6 month of storage at 35° C.

In certain embodiments, the provided lyophilized formulation retains at least 30%, preferably at least 40%, more preferably at least 50% and most preferably at least 60% C1-INH activity after 12 months of storage at 35° C.

In certain embodiments, the provided lyophilized formulation retains at least 30%, preferably at least 40%, more preferably at least 50% and most preferably at least 60% C1-INH activity after 24 months of storage at 35° C.

Hence, the provided pharmaceutical formulations can be utilized as stand-by medication, i.e., a patient suffering from e.g. hereditary angioedema can always keep such a formulation in close proximity (without the requirement of cooling) in order to have an immediate treatment available upon occurrence of an edema attack. Moreover, the provided pharmaceutical formulations demonstrate a long-term stability compared to concentrated C1-INH formulations having only a buffer excipient in low concentrations.

The provided pharmaceutical formulations are suitable for subcutaneous as well as intravenous administration. In general, subcutaneous administration is preferred upon prophylactic treatment of patients suffering from hereditary angioedema and intravenous administration is preferred upon acute treatment of patients suffering from hereditary angioedema. Neither the intravenous nor the subcutaneous administration of the provided pharmaceutical formulations causes serious drug-related adverse side events but only minor treatment-emergent adverse effects. Hence, the provided formulations can be used for both administrations. In further embodiments, the pharmaceutical formulation described herein is suitable for intra-arterial and/or intramuscular administration.

Moreover, the patients can self-administer the provided pharmaceutical formulations.

Patients can use the provided formulations for prophylactic treatment and, in addition, the same formulation can be used for an acute treatment upon occurrence of an angioedema attack. Hence, patients are only supplied with one type of formulation, which is indicative of a high patient compliance and which can be used as required. For these reasons, the provided formulations may achieve a high patient compliance.

Furthermore, the provided formulations exhibit a high local tolerance upon subcutaneous and intravenous injection; they are well tolerated, with no serious drug-related adverse events. In particular, the subcutaneous administration of the provided formulations is safe and well tolerated with only mild-to-moderate local site reactions. Likewise, a high local tolerance is achieved upon intra-arterial injection and intramuscular injection.

Moreover, thrombotic events, clot formation, thromboembolic complications do not occur after administration of the described formulations in any of the described doses in a patient. Further, administration of the described formulations does not enhance the thrombogenic risk in a patient.

The pharmaceutical formulations of the present invention ensure a good bioavailability of the C1-INH upon subcutaneous as well as upon intravenous administration. Upon subcutaneous administration, a C1-INH bioavailability in a human subject of about 40-50% (compared to intravenous administration) may be achieved. Reasons for this subcutaneous bioavailability could be attributed, in part, to degradation or consumption at the injection site or during lymphatic and blood vessel transport. It is known that C1-INH can be metabolized quickly in patients with C1-INH deficiency; this is in contrast to most other inherited plasma protein deficiencies in which heterozygous carries have 50% of normal protein levels.

Moreover, periodic subcutaneous administration of the provided formulations leads to a dose-dependent increase in functional C1-INH activity, with the 3000 IU and 6000 IU doses twice-weekly achieving constant C1-INH activity levels above 40% of the plasma level in a healthy person, which would have a clinically meaningful effect in preventing HAE attacks. Upon subcutaneous administration of the provided formulations, C1-INH functional activity time profiles with a considerably lower peak-to-trough ratio and more consistent exposures after subcutaneous administration are achieved. This lower peak-to-trough fluctuations for the subcutaneous administration are particularly desired upon prophylactic treatment, as such plasma levels ensure persistent protection from the occurrence of angioedema attacks in patients suffering from hereditary angioedema.

In another aspect, a kit is provided comprising a lyophilized formulation described herein and the respective amount of a liquid suitable for reconstitution. In certain embodiments, the suitable liquid is water for injection, preferable deionized sterile water for injection.

In some embodiments, the kit can comprise a pharmaceutical formulation described herein and a syringe. In certain embodiments, the syringe is suitable for subcutaneous injection. In other embodiments, the syringe is suitable for intravenous injection. In alternative embodiments, the syringe is suitable for subcutaneous injection and intravenous injection. In further embodiments, the syringe is suitable for intra-arterial injection and/or intramuscular injection.

In some embodiments, the kit further can comprise a needle suitable for intravenous injection and/or a needle suitable for subcutaneous injection. In a further embodiment, the kit can comprise a needle suitable for intra-arterial injection and/or a needle suitable for intramuscular injection.

In another aspect, a syringe prefilled with the liquid formulation described herein is provided. In certain embodiments, the syringe is suitable for subcutaneous injection. In other embodiments, the syringe is suitable for intravenous injection. In alternative embodiments, the syringe is suitable for subcutaneous injection and intravenous injection. In further embodiments, the syringe is suitable for intra-arterial injection and/or intramuscular injection.

### Methods and Uses

The provided formulations can be used in the treatment of various diseases and conditions.

In some embodiments, the provided formulations can be used in the treatment and/or prevention of disorders related to kinin formation, in particular hereditary angioedema (HAE), secondary brain edema, edema of the central nervous system, hypotensive shock, or edema during or after contacting blood with an artificial surface.

Further provided is a method of treating or preventing disorders related to kinin formation, in particular hereditary angioedema (HAE), secondary brain edema, edema of the central nervous system, hypotensive shock, or edema during or after contacting blood with an artificial surface in a patient, comprising administering a pharmaceutically effective dose of any of the formulations described herein.

In certain embodiments, the provided formulations are used in the treatment and/or prophylaxis of hereditary angioedema, in particular HAE type I, HAE type II and/or HAE type III.

In certain embodiments, a method of treating or preventing hereditary angioedema, in particular HAE type I, HAE type II and/or HAE type III in a patient, comprising administering a pharmaceutically effective dose of any of the formulations described herein, is provided.

In further embodiments, the provided formulations can be used in the treatment and/or prophylaxis of an ischemia-reperfusion injury (IRI), in particular wherein the IRI is due to surgical intervention, in particular vascular surgery, cardiac surgery, neurosurgery, trauma surgery, cancer surgery, orthopedic surgery, transplantation, minimally invasive surgery, or insertion of a device for delivery of a pharmacologically active substance or for mechanical removal of complete or partial obstructions.

Further provided is a method of treating or preventing an ischemia-reperfusion injury (IRI), in particular wherein the IRI is due to surgical intervention, in particular vascular surgery, cardiac surgery, neurosurgery, trauma surgery, cancer surgery, orthopedic surgery, transplantation, minimally invasive surgery, or insertion of a device for delivery of a pharmacologically active substance or for mechanical removal of complete or partial obstructions in a patient, comprising administering a pharmaceutically effective dose of any of the formulations described herein.

In some embodiments, the provided formulations can be used to prevent rejection of a transplanted tissue in a patient. The transplantation can be allotransplantation or xenotransplantation.

Further provided is a method of preventing rejection of a transplanted tissue in a patient, comprising administering a pharmaceutically effective dose of any of the formulations described herein. The transplantation can be allotransplantation or xenotransplantation.

In further embodiments, the provided formulations can be used in the treatment and/or prevention of retinopathy.

Further provided is a method of treating or preventing retinopathy in a patient, comprising administering a pharmaceutically effective dose of any of the formulations described herein.

### Treatment and Administration

In various embodiments, acute treatment occurs upon treatment of a patient having hereditary angioedema and suffering from an acute angioedema attack.

In further embodiments, prophylactic treatment of a patient suffering from hereditary angioedema occurs in order to prevent the occurrence of an edema. Prophylactic treatment of patients suffering from hereditary angioedema can be done regularly and can also occur occasionally, for example before surgical interventions, dental treatments and other symptom-triggering situations such as a situation where a patient realizes an upcoming edema.

In various embodiments, the provided formulations can be administered via subcutaneous injection. In alternative embodiments, the provided formulations can be administered via intravenous injection. The formulations can be administered continuously by infusion or by bolus injection. In certain embodiments, the provided formulations can be administered via subcutaneous injection and via intravenous injection. In further embodiments, the patient can self-administer the provided formulations.

In some embodiments, the provided formulation is administered via intravenous injection during acute treatment of a patient. In other embodiment, the provided formulation is administered via subcutaneous injection during prophylactic treatment of a patient.

In further embodiments, the provided formulations can be administered via intra-arterial injection. In further embodiments, the provided formulations can be administered via intramuscular injection.

In further embodiments, the formulations described herein may be administered to a patient by any pharmaceutically suitable means of administration. Various delivery systems are known and can be used to administer the composition by any convenient route. Preferentially the formulations of the invention are administered systemically. For systemic use, the therapeutic proteins of the invention are formulated for parenteral (e.g. intravenous, subcutaneous, intramuscular, intraperitoneal, intracerebral, intrapulmonar, intranasal or transdermal) or enteral (e.g., oral, vaginal or rectal) delivery according to conventional methods. Some formulations encompass slow release systems.

In some embodiments, the provided pharmaceutical formulations have an kinematic viscosity of the formulation as determined immediately after preparation above the values of pure water (about 1 mm²/s) but below 10 mm²/s, preferably below 8 mm²/s and more preferably below 6 mm²/s and even more preferably below 5 mm²/s. Determination of the kinematic viscosity is done at +20° C. In certain embodiments, said kinematic viscosities apply to solutions comprising about 400-625 IU/mL C1-INH, preferably about 400-600 IU/mL, more preferably about 450-550 IU/mL. The lower the viscosity, the lower the required force for delivering a specified volume of the drug within a certain time frame, which is an advantage for applying the drug both intravenously and subcutaneously. This is particularly advantageous when patients self-administer C1-INH formulations.

### Dosing Schemes

In certain embodiments, a dose of C1-INH of about 1,200 IU, about 1,500 IU, about 1,600 IU, about 1,700 IU, about 1,800 IU, about 1,900 IU, about 2,000 IU, about 2,100 IU, about 2,200 IU, about 2,300 IU, about 2,400 IU, about 2,500 IU, about 2,600 IU, about 2,700 IU, about 2,800 IU, about 2,900 IU, about 3,000 IU, about 3,500 IU, about 4,000 IU, about 4,500 IU, about 5,000 IU, about 5,500 IU or about 6,000 IU or any amount in between is administered to a patient. In preferred embodiments, a dose of about 1,500 IU, about 2,000 IU, about 3,000 IU, about 4,000 IU, about 5,000 IU or about 6,000 IU is administered to a patient.

In further embodiments, a dose of C1-INH of about 10-100 IU/kg bodyweight, of about 20-90 IU/kg bodyweight, of about 20-80 IU/kg bodyweight, of about 30-70 IU/kg bodyweight, of about 40-60 IU/kg bodyweight or any range in between is administered to a patient.

In certain embodiments, upon subcutaneous administration a dose of C1-INH of about 20-80 IU/kg bodyweight, of about 30-80 IU/kg bodyweight, of about 40-80 IU/kg bodyweight, of about 40-60 IU/kg bodyweight, of about 50-60 IU/kg bodyweight or any range in between is administered to a patient.

In certain embodiments, upon intravenous administration, preferably in the acute treatment, a dose of C1-INH of about 10-60 IU/kg bodyweight, of about 20-40 IU/kg bodyweight, of about 20 IU/kg bodyweight or any range in between is administered to a patient.

In certain embodiments the target is to reach a mean C1-INH activity level in the prophylactic treatment.

In various embodiments, a dose of C1-INH is administered, preferably in prophylactic treatment, at intervals of 1, 2, 3, 4, 5, 6 or 7 days. In further embodiments, a dose of C1-INH is administered, preferably in prophylactic treatment, at intervals of every 1-2 days, at intervals of every 2-3 days, at intervals of every 3-4 days, at intervals of every 4-5 days, at intervals of every 5-6 days or at intervals of every 6-7 days.

In some embodiments, a dose comprising one, two or more FDF(s) of about 1,500 IU of the C1-INH formulation is/are administered, preferably subcutaneously, at intervals of every 2-3 days.

In further embodiments, a dose comprising one, two or more FDF(s) of about 3,000 IU of the C1-INH formulation is/are administered, preferably subcutaneously, at intervals of every 3-4 days.

In further embodiments, a dose comprising about 1,500 IU, about 3,000 IU, about 4,000 IU, about 5,000 IU or about 6,000 IU of the C1-INH formulation is/are administered, preferably subcutaneously, weekly.

In further embodiments, a dose comprising about 1,500 IU, about 3,000 IU, about 4,000 IU, about 5,000 IU or about 6,000 IU of the C1-INH formulation is/are administered, preferably subcutaneously, twice-weekly.

In other embodiments, a dose comprising a dose of about 20-40 IU C1-INH per kg bodyweight is administered, preferably subcutaneously, at intervals of every 2-3 days.

In further embodiments, a dose comprising a dose of about 40-60 IU C1-INH per kg bodyweight is administered, preferably subcutaneously, at intervals of every 3-4 days.

In further embodiments, a dose comprising a dose of about 40-80 IU C1-INH per kg bodyweight or of about 40-60 IU C1-INH per kg bodyweight is administered, preferably subcutaneously, weekly.

In further embodiments, a dose comprising a dose of about 40-80 IU C1-INH per kg bodyweight or of about 40-60 IU C1-INH per kg bodyweight is administered, preferably subcutaneously, twice weekly.

In other embodiments, one, two or more FDF(s) of about 1,500 IU C1-INH is/are administered to a patient during acute treatment, preferably via intravenous administration. In further embodiments, one or more FDF(s) of about 3,000 IU C1-INH is/are administered to a patient during acute treatment, preferably via intravenous administration.

In further embodiments, one, two or more dose(s) of about 20 IU C1-INH per kg bodyweight is/are administered to a patient during acute treatment, preferably via intravenous administration. In further embodiments, one or more dose(s) of about 40 IU C1-INH per kg bodyweight is/are administered to a patient during acute treatment, preferably via intravenous administration.

The Figures show:

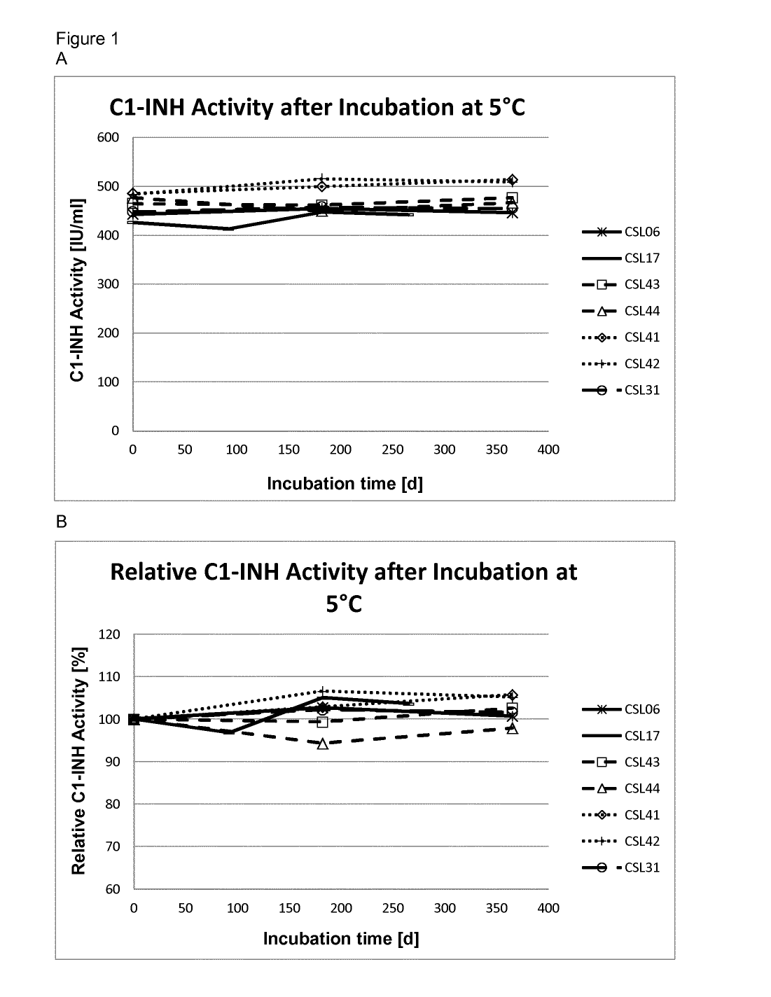

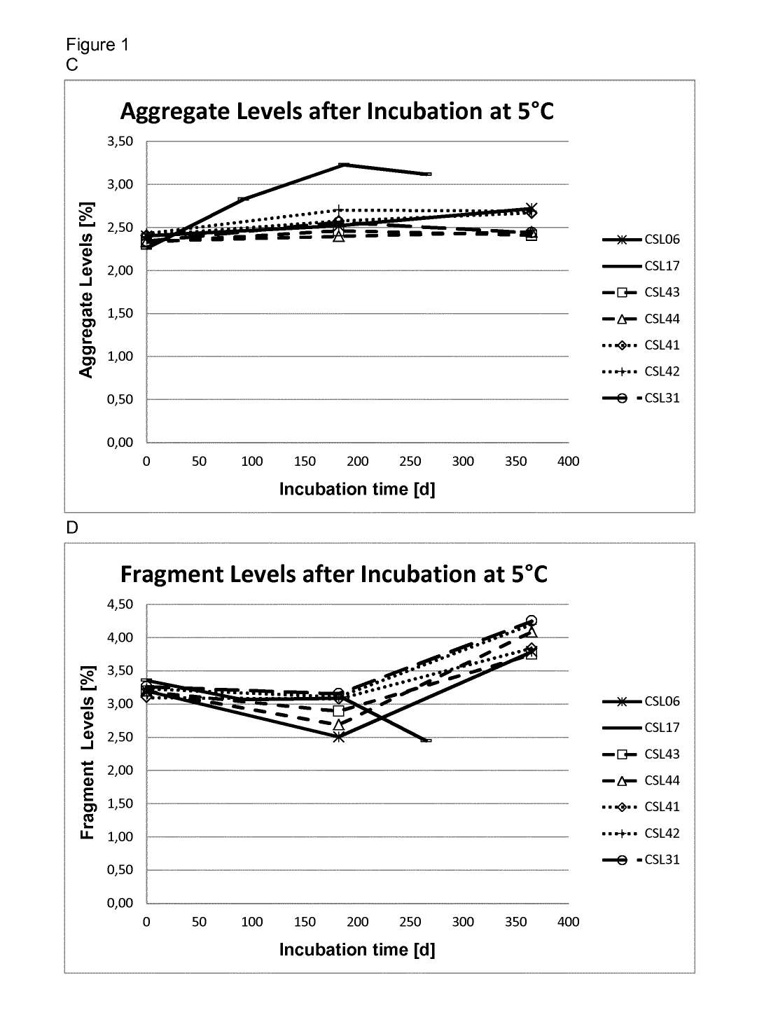

FIG. 1: C1-INH activity (1a), relative C1-INH activity (1b), HMWC levels by SEC HPLC (1c) and fragment levels by SEC HPLC (1d) of different C1-INH formulations after incubation at 5° C.

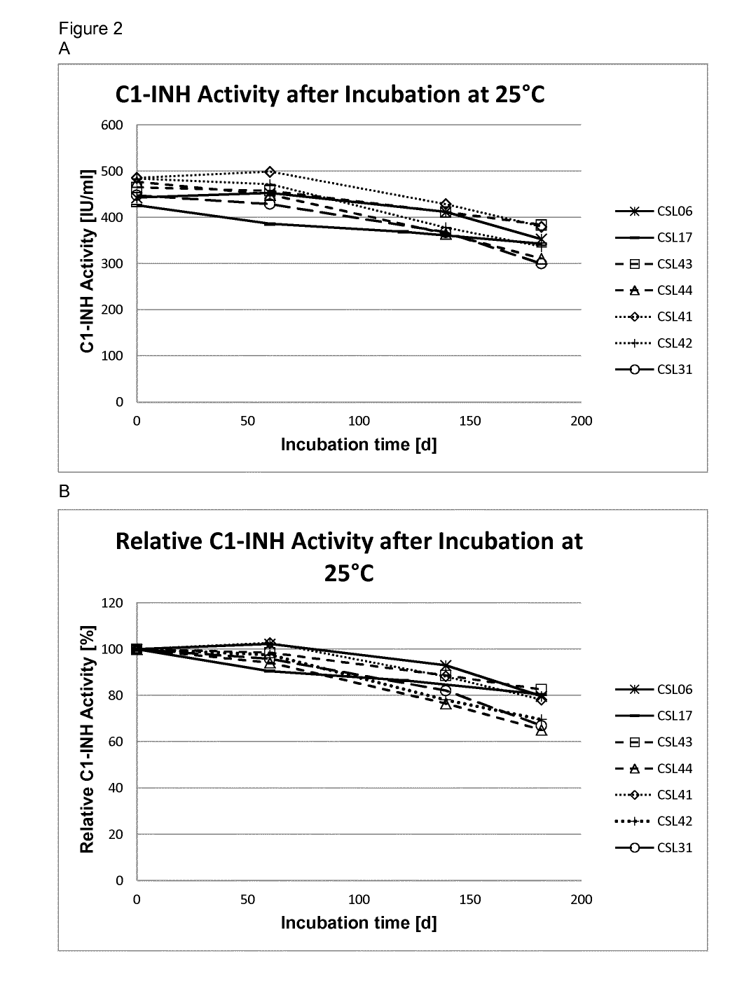

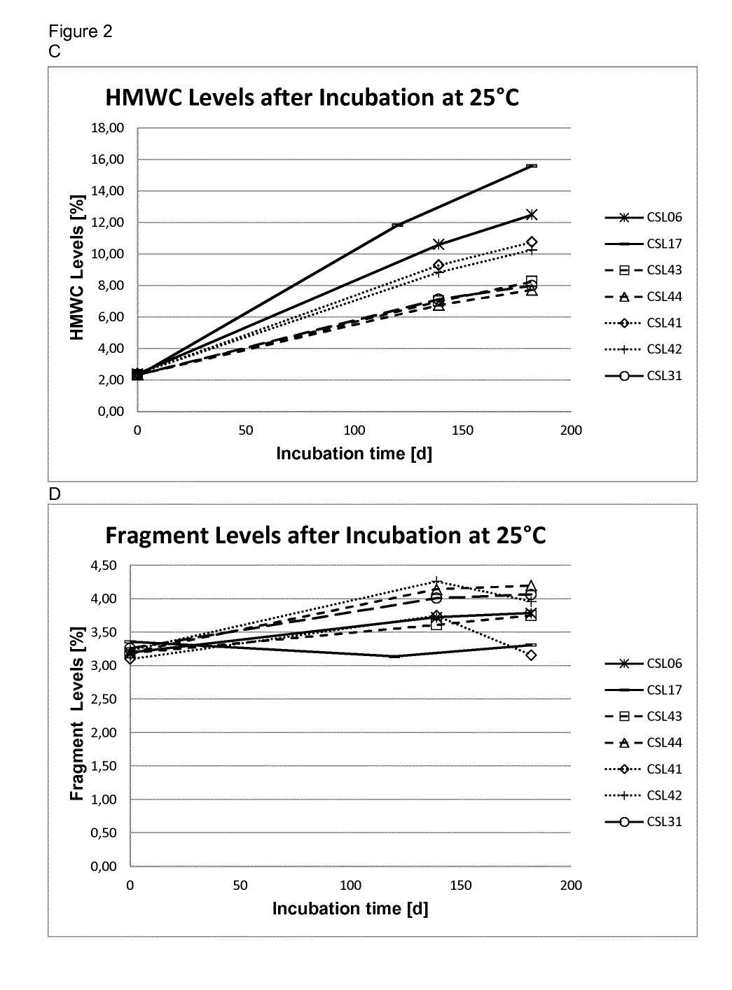

FIG. 2: C1-INH activity (2a), relative C1-INH activity (3b), HMWC levels by SEC HPLC (2c) and fragment levels by SEC HPLC (2d) of different C1-INH formulations after incubation at 25° C.

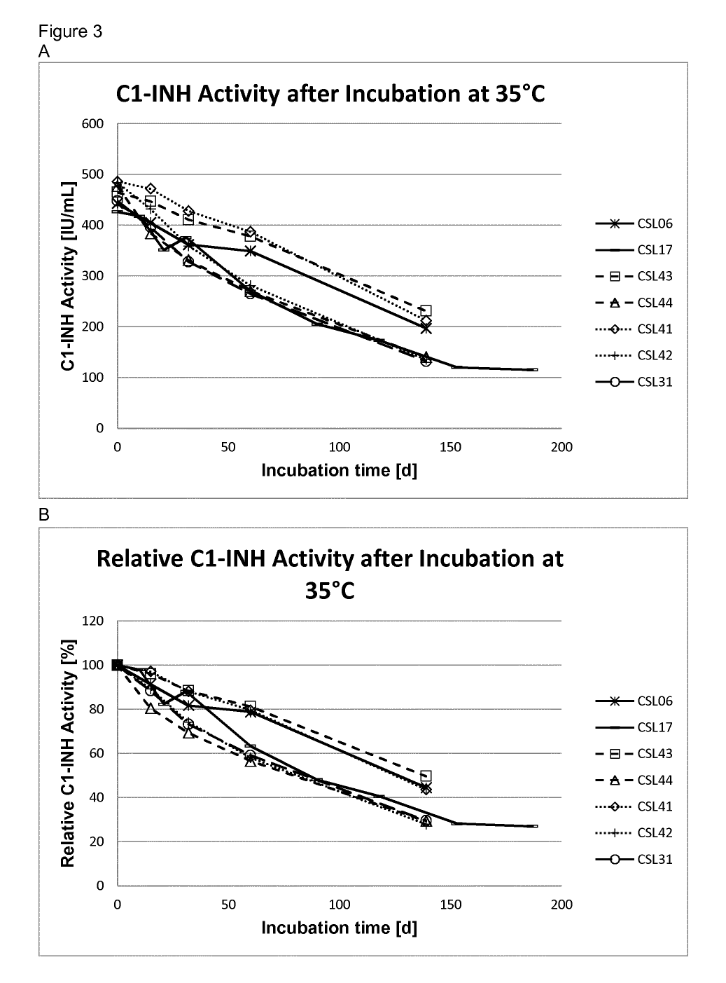

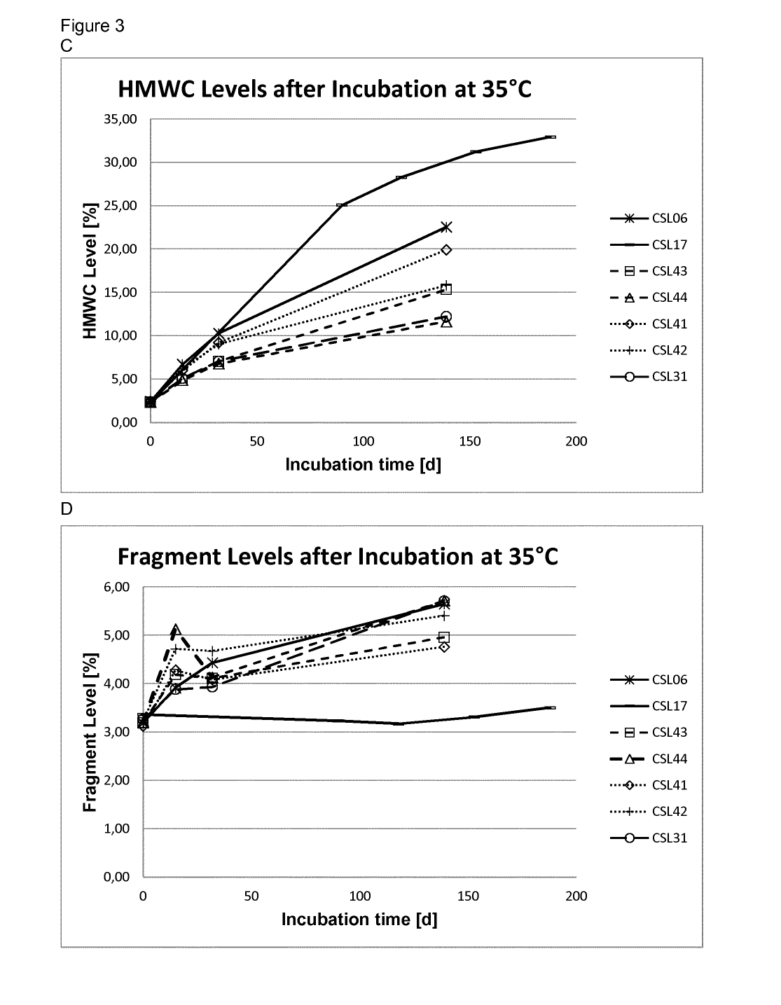

FIG. 3: C1-INH activity (3a), relative C1-INH activity (3b), HMWC levels by SEC HPLC (3c) and fragment levels by SEC HPLC (3d) of different C1-INH formulations after incubation at 35° C.

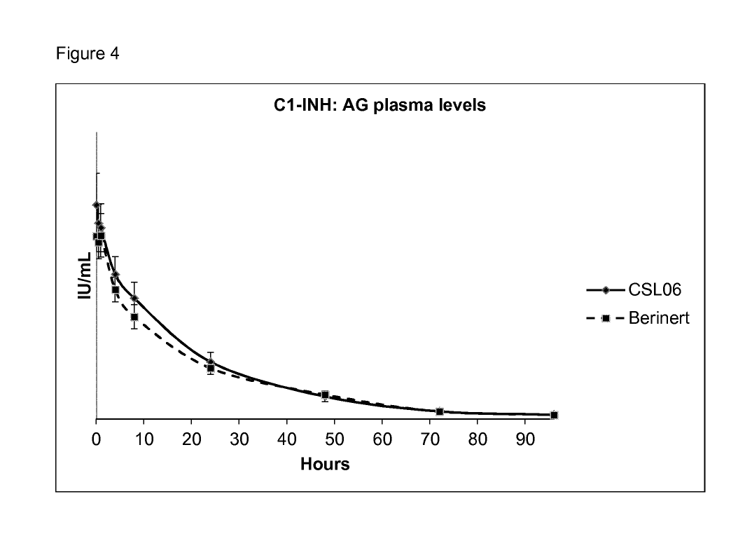

FIG. 4: Mean values of C1-INH:Ag plasma levels (linear scale) showing the bioequivalence between Berinert® and CSL06. The sample size was N=10 for each time point and group, save the 96 hour value of the CSL06 group (n=2).

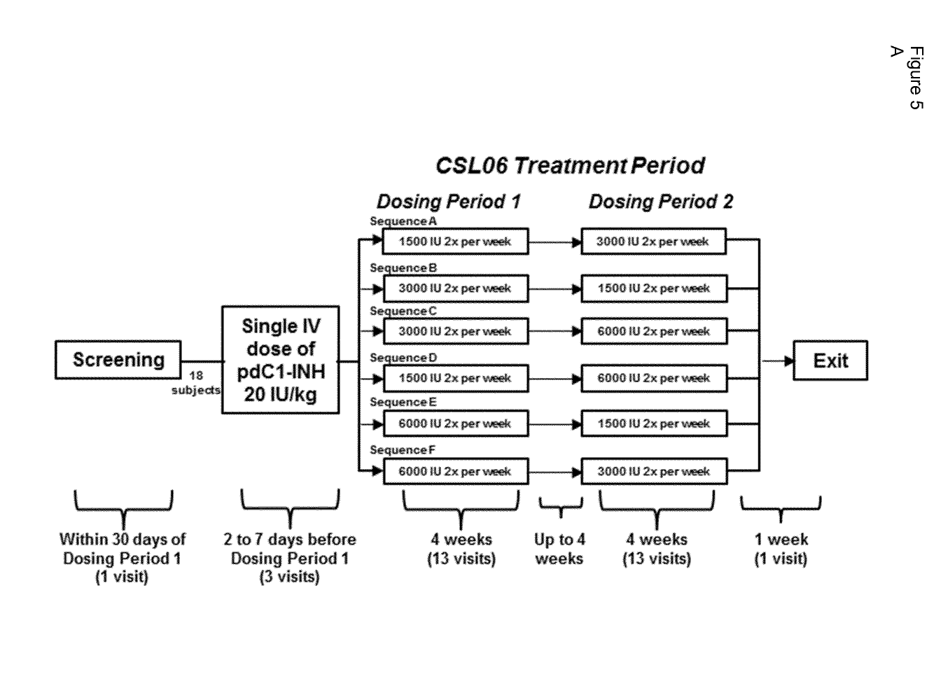

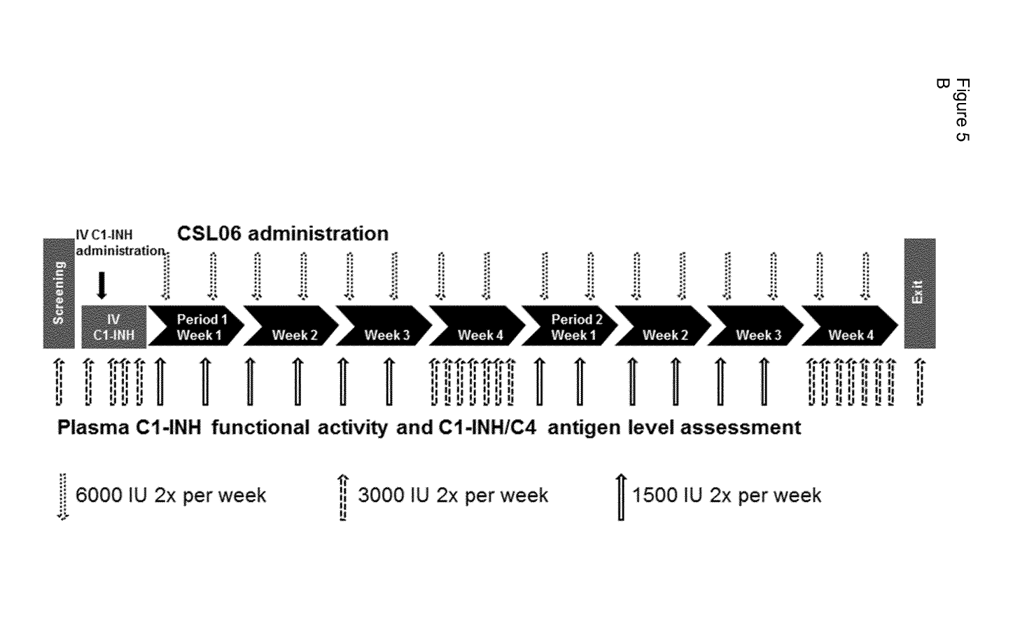

FIG. 5: Study schema. (a) dosing scheme and sample collection during the study, (b) [symbol] =single dose of IV C1-INH; [symbol] =single dose of SC CSL06; [symbol] =assessment of C1-INH functional activity and plasma C1-INH and C4 antigen concentrations; [symbol] =additional assessments of plasma C1-INH functional activity.

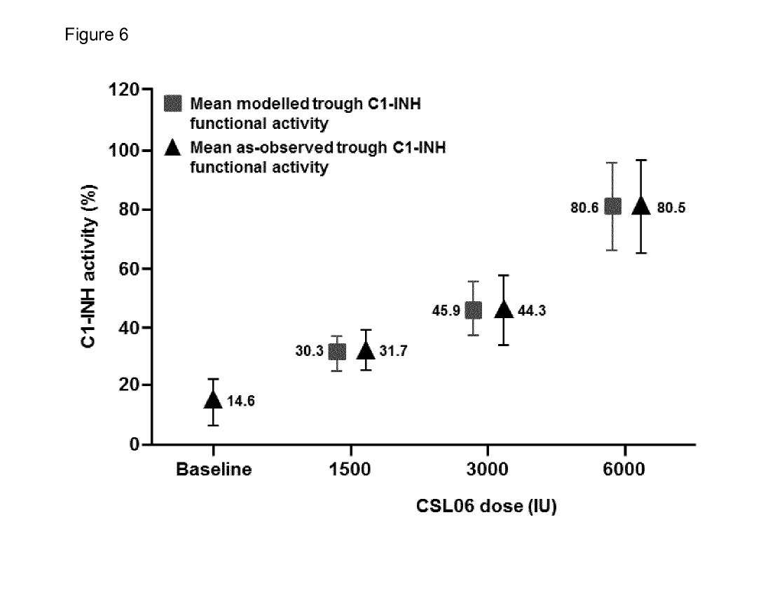

FIG. 6: PK results of CSL06. Modeled steady-state trough C1-INH functional activity (primary endpoint; red rectangles) and as-observed C1-INH functional activity (black triangles). Data points show the mean and 95% Cl.

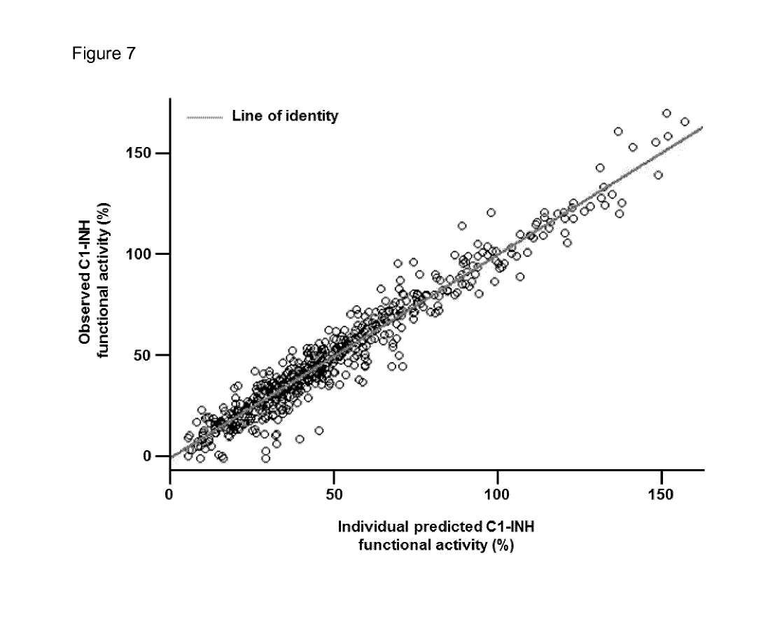

FIG. 7: Final population PK model of as-observed C1-INH functional activity versus individual predictions of C1-INH functional activity. The line of identity (solid) is included as a reference.

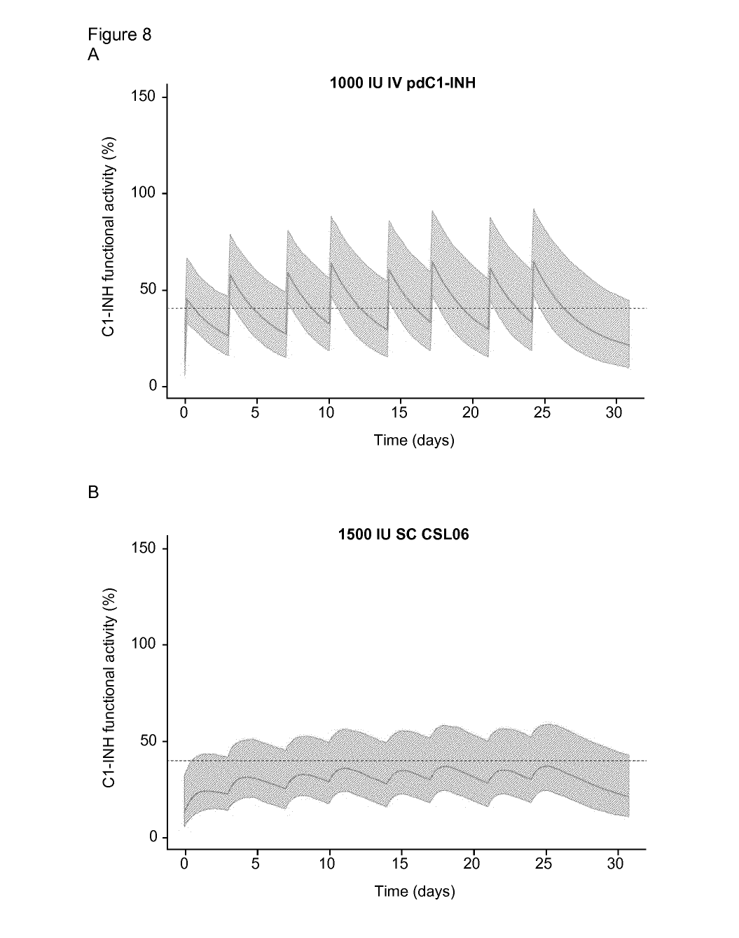

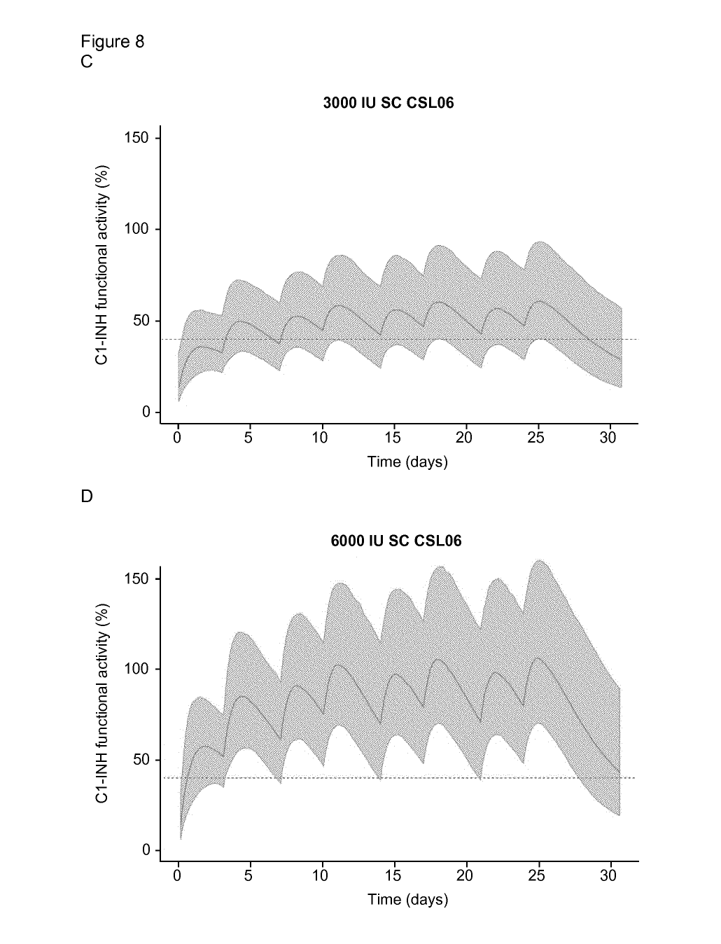

FIG. 8: Modeled biweekly C1-INH functional activity after IV administration of (a) a therapeutic dose of 1000 IU pd C1-INH concentrate, or SC administration of (b) 1500 IU, (b) 3000 IU, or (c) 6000 IU of CSL06. Median functional activity (solid lines), 5th and 95th percentiles (shaded areas) and 40% C1-INH functional activity (dashed line) are shown.

### EXAMPLES

The examples illustrate the present invention while in no way limiting it.

### Example 1: Generation of C1-INH Formulations

A C1-INH concentrate, manufactured based on the Berinert® manufacturing process (according to A. Feussner et al., Transfusion 2014, 54: 2566-73) but concentrated to an increased C1-INH concentration (after reconstitution), is used to prepare several C1-INH formulations. The different C1-INH formulations are prepared by dissolving the freeze dried C1-INH concentrate in WFI at a concentration of about 1500 IU/ml. PD10 desalting columns (GE Healthcare) are equilibrated with the target formulation buffer containing the respective excipients at the mentioned concentrations (Table 1). Then a small amount of the C1-INH solution is applied per PD10 column. C1-INH is eluted with the corresponding formulation buffer within the column exclusion volume. C1-INH concentration is determined by chromogenic assay (Berichrom C1-Inhibitor, Siemens) and adjusted to the respective concentration using the corresponding formulation buffer.

TABLE 1
Excipients and their concentration of different C1-INH formulations and the calculated osmolarity.

| | | Na₃- | Na₂H/NaH₂- | | Arginine * | | | Na₂- | Na- | | Calculated |
|---|---|---|---|---|---|---|---|---|---|---|---|
| | C1-INH | Citrate | Phosphate | Glycine | HCl | NaCl | Na₂SO₄ | Succinate | Acetate | PS80 | osmolarity |
| | [IU/mL] | [mM] | [mM] | [mM] | [mM] | [mM] | [mM] | [mM] | [mM] | [mg/mL] | [mOsm/L] |
| Berinert ® | 50 | 13 | | 133 | | 154 | | | | | 493 |
| CSL01 | 500 | 10 | | 133 | | 145 | | | | | 463 |
| CSL02 | 500 | 10 | | 133 | | 145 | | | | 1 | 463 |
| CSL03 | 450 | 13 | | 133 | | 154 | | | | | 493 |
| CSL04 | 450 | 10 | | 133 | | 154 | | | | | 481 |
| CSL05 | 500 | 7 | | 133 | | 154 | | | | | 469 |
| CSL06 | 500 | 13 | | 133 | | 154 | | | | | 493 |
| CSL07 | 500 | 13 | | 133 | | 154 | | | | 1 | 493 |
| CSL08 | 500 | 20 | | 133 | | 154 | | | | | 521 |
| CSL09 | 500 | 25 | | 133 | | 154 | | | | | 541 |
| CSL10 | 500 | 30 | | 133 | | 154 | | | | | 561 |
| CSL11 | 550 | 10 | | 133 | | 154 | | | | | 481 |
| CSL12 | 550 | 25 | | 133 | | 154 | | | | | 541 |
| CSL13 | 600 | 10 | | 133 | | 154 | | | | | 481 |
| CSL14 | 600 | 25 | | 133 | | 154 | | | | | 541 |
| CSL15 | 750 | 10 | | 133 | | 154 | | | | | 481 |
| CSL16 | 750 | 25 | | 133 | | 154 | | | | | 541 |
| CSL17 | 500 | 17 | | 133 | | 68 | | | | | 337 |
| CSL18 | 500 | 17 | | 133 | | 68 | | | | 1 | 337 |
| CSL19 | 500 | 10 | | | | | | | 225 | | 490 |
| CSL20 | 500 | 10 | | | | | | | 225 | 1 | 490 |
| CSL21 | 500 | 10 | | | | | | 150 | | | 490 |
| CSL22 | 500 | 10 | | | | | | 150 | | 1 | 490 |
| CSL23 | 500 | 10 | | | 225 | | | | | | 490 |
| CSL24 | 500 | 10 | | | 225 | | | | | 1 | 490 |
| CSL25 | 500 | 10 | | | | 225 | | | | | 490 |
| CSL26 | 500 | 10 | | | | 225 | | | | 1 | 490 |
| CSL27 | 500 | 10 | | | | 100 | 83 | | | | 489 |
| CSL28 | 500 | 10 | | | | 100 | 83 | | | 1 | 489 |
| CSL29 | 500 | 10 | | | | | 50 | 40 | | | 310 |
| CSL30 | 500 | 10 | | | | | 50 | 40 | | 1 | 310 |
| CSL31 | 500 | 10 | | | | | 83 | 67 | | 1 | 490 |
| CSL32 | 500 | 25 | | | | | 83 | 67 | | | 550 |
| CSL33 | 550 | 10 | | | | | 83 | 67 | | | 490 |
| CSL34 | 600 | 10 | | | | | 83 | 67 | | | 490 |
| CSL35 | 750 | 10 | | | | | 83 | 67 | | | 490 |
| CSL36 | 500 | 10 | | | | | 83 | 67 | | 1 | 490 |
| CSL37 | 500 | 10 | | | 24 | | 50 | 24 | | | 310 |
| CSL38 | 500 | 10 | | | 24 | | 50 | 24 | | 1 | 310 |
| CSL39 | 500 | 10 | | | 40 | | 83 | 40 | | | 489 |
| CSL40 | 500 | 10 | | | 40 | | 83 | 40 | | 1 | 489 |
| CSL41 | 500 | 10 | | | | | 90 | | | | 310 |
| CSL42 | 500 | 10 | | | | | 90 | | | 1 | 310 |
| CSL43 | 500 | 10 | | | | | 150 | | | | 490 |
| CSL44 | 500 | 10 | | | | | 150 | | | 1 | 490 |
| CSL45 | 500 | 10 | | | 34 | | 34 | 34 | | | 312 |
| CSL46 | 500 | 25 | | | 34 | | 34 | 34 | | | 372 |
| CSL47 | 550 | 10 | | | 34 | | 34 | 34 | | | 312 |
| CSL48 | 600 | 25 | | | 34 | | 34 | 34 | | | 372 |
| CSL49 | 750 | 10 | | | 34 | | 34 | 34 | | | 312 |
| CSL50 | 500 | 10 | | | 34 | | 34 | 34 | | 1 | 312 |
| CSL51 | 500 | 10 | | | 57 | | 57 | 57 | | | 496 |
| CSL52 | 500 | 25 | | | 57 | | 57 | 57 | | | 556 |
| CSL53 | 550 | 10 | | | 57 | | 57 | 57 | | | 496 |
| CSL54 | 600 | 10 | | | 57 | | 57 | 57 | | | 496 |
| CSL55 | 600 | 25 | | | 57 | | 57 | 57 | | | 556 |
| CSL56 | 500 | 10 | | | 57 | | 57 | 57 | | 1 | 496 |
| CSL57 | 500 | 10 | | | | 60 | 50 | | | | 310 |
| CSL58 | 500 | 10 | | | | 60 | 50 | | | 1 | 310 |
| CSL59 | 500 | | 24 | | | 225 | | | | | 510 |
| CSL60 | 500 | | 36 | | | 225 | | | | | 540 |
| CSL61 | 450 | | 24 | | | 225 | | | | | 510 |
| CSL62 | 550 | | 24 | | | 225 | | | | | 510 |
| CSL63 | 600 | | 24 | | | 225 | | | | | 510 |
| CSL64 | 600 | | 36 | | | 225 | | | | | 540 |
| CSL65 | 750 | | 24 | | | 225 | | | | | 510 |
| CSL66 | 500 | | 24 | | | 135 | | | | | 330 |
| CSL67 | 550 | | 24 | | | 135 | | | | | 330 |
| CSL68 | 500 | | 36 | | | 135 | | | | | 360 |
| CSL69 | 500 | | 24 | | | | 150 | | | | 510 |
| CSL70 | 500 | | 36 | | | | 150 | | | | 540 |
| CSL71 | 450 | | 24 | | | | 150 | | | | 510 |
| CSL72 | 550 | | 24 | | | | 150 | | | | 510 |
| CSL73 | 600 | | 24 | | | | 150 | | | | 510 |
| CSL74 | 600 | | 36 | | | | 150 | | | | 540 |
| CSL75 | 750 | | 24 | | | | 150 | | | | 510 |
| CSL76 | 500 | | 24 | | | | 90 | | | | 330 |
| CSL77 | 550 | | 24 | | | | 90 | | | | 330 |
| CSL78 | 500 | | 36 | | | | 90 | | | | 360 |

### Example 2: Determination of the Melting Temperature of C1-INH by DSF

For determination of the melting temperature of C1-INH by DSF a C1-INH concentrate (manufactured based on the Berinert® manufacturing process according to A. Feussner et al., Transfusion 2014, 54: 2566-73) buffered in various buffers was mixed with stock solutions of salts and/or other excipients, water and Sypro Orange (Life Technologies) in order to obtain a concentration of C1-INH of 36 IU/ml, the final concentrations of buffers, salts and/or other excipients as listed in Tables 2 and 3 and a dilution of the original Sypro Orange stock solution of 1:1000. The total volume per assay reaction was 50 μl. As the DSF assay is used primarily in optimizing the type and concentration of the excipients of a protein formulation (instead of the concentration of that protein), the used C1-INH concentration is optimized for the efficiency of this assay.

The differential scanning fluorimetry (DSF) was performed in a 96 well multiplate on a Real Time Cycler (Applied Biosystems 7500). Temperature was increased from 25° C. to 95° C. with a ramp of 1° C./min. Fluorescence was continually recorded at 610 nm emission wave length. Data were analyzed using the Protein Thermal Shift Software (Life Technologies). The melting temperature of C1-INH was determined as the maximum of the first derivative of the fluorescence plotted against temperature. Results for the melting temperature measured by DSF are shown in Tables 2 and 3.

### Results

The data indicate that the melting temperature by DSF is much lower for formulations having only the excipient phosphate or citrate in low concentrations in their buffer. The same applies to formulations where additional uncharged excipients such as proline and saccharose are present although such formulations have an overall calculated osmolarity of about 300 mOsm/L. In contrast, formulations having a low concentration of citrate or phosphate and having an additional amount of physiological acceptable salt(s) such as NaCl, sodium sulphate and/or sodium succinate and an overall calculated osmolarity of about 300 to 500 mOsml/L have a melting temperature measured by DSF of 55° C. or higher and appear to be stable based on these data (and the long term stability data, see example 3).

TABLE 2
Osmolarity and stability measurements of C1-INH formulations (pH = 7.2) by using melting temperature (mt) determination by DSF.

| Na₃- | Na₂H/NaH₂- | | | | Na₂- | Na- | | | Arginine * | Na- | | calculated |
|---|---|---|---|---|---|---|---|---|---|---|---|---|
| Citrate | Phosphate | NaCl | Glycine | Na₂SO₄ | Succinate | Acetate | L-Proline | Saccarose | HCl | Glutamate | DSF mt | Osmolarity |
| [mM] | [mM] | [mM] | [mM] | [mM] | [mM] | [mM] | [mM] | [mM] | [mM] | [mM] | [° C.] | [mOsm/L] |
| | 8 | | | | | | | | | | 42.5 | 20 |
| | 17 | | | | | | | | | | 47.0 | 42.5 |
| | 17 | | | | | | 250 | | | | 47.5 | 293 |
| | 17 | | | | | | | 250 | | | 49.5 | 293 |
| | 24 | | | | | | | | | | 50.0 | 60 |
| | 34 | | | | | | | | | | 51.0 | 85 |
| | 24 | | | | | | 250 | | | | 50.0 | 310 |
| | 24 | | | | | | | 250 | | | 51.5 | 310 |
| | 24 | 135 | | | | | | | | | 55.5 | 330 |
| | 24 | 225 | | | | | | | | | 56.5 | 510 |
| | 24 | | | 90 | | | | | | | 57.0 | 330 |
| | 24 | | | 150 | | | | | | | 58.0 | 510 |
| 10 | | | | | | | | | | | 48.5 | 40 |
| 20 | | | | | | | | | | | 52.0 | 80 |
| 40 | | | | | | | | | | | 53.0 | 160 |
| 13 | | 154 | 133 | | | | | | | | 55.0 | 493 |
| 10 | | | | | | | | | | 130 | 55.2 | 300 |
| 10 | | 135 | | | | | | | | | 55.5 | 310 |
| 10 | | | | | | | | | 225 | | 56.2 | 490 |
| 10 | | | | | | | | | | 230 | 56.2 | 500 |
| 10 | | | | 50 | 24 | | | | 24 | | 56.3 | 310 |
| 10 | | | | | | 225 | | | | | 56.6 | 490 |
| 10 | | 225 | | | | | | | | | 56.6 | 490 |
| 10 | | | | 34 | 34 | | | | 34 | | 56.7 | 312 |
| 10 | | | | 90 | | | | | | | 57.0 | 310 |
| 10 | | 100 | | 83 | | | | | | | 57.4 | 489 |
| 10 | | | | 57 | 57 | | | | 57 | | 57.7 | 496 |
| 10 | | | | 83 | 40 | | | | 40 | | 57.9 | 489 |
| 10 | | | | | 150 | | | | | | 58.0 | 490 |
| 10 | | | | 150 | | | | | | | 58.0 | 490 |

TABLE 3
Osmolarity and stability measurements of C1-INH formulations
by using melting temperature (mt) determination by DSF.

| | Na₃- | | | | Na₂- | | | calculated | |
|---|---|---|---|---|---|---|---|---|---|
| | Citrate | NaCl | Glycine | Na₂SO₄ | Succinate | PS80 | DSF mt | Osmolarity | |
| | [mM] | [mM] | [mM] | [mM] | [mM] | [g/L] | [° C.] | [mOsm/L] | pH |
| CSL06 | 13 | 154 | 133 | | | | 55.3 | 483 | 7.0 |
| CSL17 | 17 | 68 | 133 | | | | 55.5 | 337 | 7.3 |
| CSL43 | 10 | | | 150 | | | 58.2 | 490 | 7.2 |
| CSL44 | 10 | | | 150 | | 1 | 59.6 | 490 | 7.2 |
| CSL41 | 10 | | | 90 | | | 56.9 | 310 | 7.2 |
| CSL42 | 10 | | | 90 | | 1 | 57.9 | 310 | 7.2 |
| CSL31 | 10 | | | 83 | 67 | 1 | 60.0 | 490 | 7.2 |

### Example 3: Long Term Stability of C1-INH Upon Liquid Storage

Freeze dried C1-INH concentrate (manufactured based on the Berinert® manufacturing process (according to A. Feussner et al., Transfusion 2014, 54: 2566-73)) was dissolved in WFI at a concentration of about 1500 IU/ml. PD10 desalting columns (GE Healthcare) were equilibrated with the target formulation buffer. Then 2.5 ml of C1-INH 1500 IU/ml were applied per PD10 column. C1-INH was eluted with 3.5 ml of the corresponding formulation buffer (see Table 3) within the column exclusion volume. C1-INH concentration was determined by chromogenic assay (Berichrom C1-Inhibitor, Siemens) and adjusted to 500 IU/ml using the corresponding formulation buffer. Formulated C1-INH was filtered through 0.2 μm and then dispensed in 0.25 ml aliquots to 0.3 ml glass vials which were then stoppered with silicon stoppers under aseptic conditions. This was done for each of the seven different C1-INH formulations which are disclosed in Table 3. The respective glass vials were finally transferred to tempered rooms at 5° C., 25° C. or 35° C. Sample vials of each C1-INH formulation were taken at various time points and tested for C1-INH activity by chromogenic assay and for HMWC formation and fragmentation by SEC HPLC on a TSK-Gel G3000SWXL (Tosoh) using 20 mM NaH₂PO₄, 20 mM Na₂HPO₄, 100 mM NaCl, pH 7.2 as eluent buffer.

### Results

Results are demonstrated in Tables 4 to 12 and FIGS. 1 to 3. These data indicate for the hyperosmolar sodium sulphate formulations (CSL43, CSL44) the best C1-INH activity conservation and the lowest HMWC formation. The addition of PS80 has a positive effect on the amount of HMWC in long term storage but shows a negative effect on the C1-INH activity conservation. The isoosmolar sodium sulphate formulations (CSL41, CSL42) like the hyperosmolar sodium chloride/glycine formulation (CSL06) have similar C1-INH activity conservation and comparable HMWC formation. In general, the formulations according to the invention show acceptable long term stabilities based on these data.

TABLE 4
C1-INH activity of different C1-INH formulations at
t₀ (start) and after incubation at 5° C. at t₁ (92 days), t₂
(182 days), t₃ (265 days) and t₄ (365 days)

| | at t₀ | at t₁ | at t₂ | at t₃ | at t₄ |
|---|---|---|---|---|---|
| | [IU/mL] | [IU/mL] | [IU/mL] | [IU/mL] | [IU/mL] |
| CSL06 | 443 | n.d. | 455 | n.d. | 446 |
| CSL17 | 426 | 413 | 448 | 442 | n.d. |
| CSL43 | 465 | n.d. | 462 | n.d. | 477 |
| CSL44 | 477 | n.d. | 450 | n.d. | 467 |
| CSL41 | 486 | n.d. | 500 | n.d. | 514 |
| CSL42 | 484 | n.d. | 516 | n.d. | 509 |
| CSL31 | 448 | n.d. | 458 | n.d. | 455 |

n.d. = not determined at that time point

TABLE 5
HMWC levels [% of total protein] of different C1-INH formulations
by SEC HPLC at t₀ (start) and after incubation at 5° C. at t₁ (92 days),
t₂ (182 days), t₃ (265 days) and t₄ (365 days)

| | at t₀ | at t₁ | at t₂ | at t₃ | at t₄ |
|---|---|---|---|---|---|
| CSL06 | 2.40 | n.d. | 2.52 | n.d. | 2.72 |
| CSL17 | 2.26 | 2.83 | 3.23 | 3.12 | n.d. |
| CSL43 | 2.33 | n.d. | 2.46 | n.d. | 2.41 |
| CSL44 | 2.34 | n.d. | 2.40 | n.d. | 2.45 |
| CSL41 | 2.41 | n.d. | 2.58 | n.d. | 2.67 |
| CSL42 | 2.43 | n.d. | 2.71 | n.d. | 2.69 |
| CSL31 | 2.34 | n.d. | 2.56 | n.d. | 2.44 |

n.d. = not determined at that time point

TABLE 6
Fragment levels [% of total protein] of different C1-INH
formulations by SEC HPLC at t₀ (start) and after incubation at 5° C. at t₁
(92 days), t₂ (182 days), t₃ (265 days) and t₄ (365 days)

| | at t₀ | at t₁ | at t₂ | at t₃ | at t₄ |
|---|---|---|---|---|---|
| CSL06 | 3.20 | n.d. | 2.51 | n.d. | 3.79 |
| CSL17 | 3.36 | 3.07 | 3.09 | 2.45 | n.d. |
| CSL43 | 3.19 | n.d. | 2.89 | n.d. | 3.75 |
| CSL44 | 3.20 | n.d. | 2.69 | n.d. | 4.09 |
| CSL41 | 3.10 | n.d. | 3.08 | n.d. | 3.84 |
| CSL42 | 3.23 | n.d. | 3.11 | n.d. | 4.20 |
| CSL31 | 3.26 | n.d. | 3.16 | n.d. | 4.25 |

n.d. = not determined at that time point

TABLE 7
C1-INH activity of different C1-INH formulations at
t₀ (start) and after incubation at 25° C. at t₁ (60 days), t₂
(120 days), t₃ (139 days) and t₄ (182 days)

| | at t₀ | at t₁ | at t₂ | at t₃ | at t₄ |
|---|---|---|---|---|---|
| | [IU/mL] | [IU/mL] | [IU/mL] | [IU/mL] | [IU/mL] |
| CSL06 | 443 | 453 | n.d. | 412 | 353 |
| CSL17 | 426 | 386 | 369 | n.d. | 343 |
| CSL43 | 465 | 458 | n.d. | 412 | 384 |
| CSL44 | 477 | 449 | n.d. | 365 | 311 |
| CSL41 | 486 | 499 | n.d. | 429 | 380 |
| CSL42 | 484 | 472 | n.d. | 378 | 337 |
| CSL31 | 448 | 429 | n.d. | 368 | 300 |

n.d. = not determined at that time point

TABLE 8
HMWC levels [% of total protein] of different C1-INH
formulations by SEC HPLC at t₀ (start) and after incubation at 25° C.
at t₁ (120 days), t₂ (139 days) and t₃ (182 days)

| | at t₀ | at t₁ | at t₂ | at t₃ |
|---|---|---|---|---|
| CSL06 | 2.40 | n.d. | 10.61 | 12.50 |
| CSL17 | 2.26 | 11.83 | n.d. | 15.60 |
| CSL43 | 2.33 | n.d. | 7.01 | 8.26 |
| CSL44 | 2.34 | n.d. | 6.77 | 7.73 |
| CSL41 | 2.41 | n.d. | 9.30 | 10.77 |
| CSL42 | 2.43 | n.d. | 8.84 | 10.26 |
| CSL31 | 2.34 | n.d. | 7.13 | 7.99 |

n.d. = not determined at that time point

TABLE 9
Fragment levels [% of total protein] of different C1-
INH formulations by SEC HPLC at t₀ (start) and after incubation at 25°
C. at t₁ (120 days), t₂ (139 days) and t₃ (182 days)

| | at t₀ | at t₁ | at t₂ | at t₃ |
|---|---|---|---|---|
| CSL06 | 3.20 | n.d. | 3.73 | 3.79 |
| CSL17 | 3.36 | 3.14 | n.d. | 3.31 |
| CSL43 | 3.19 | n.d. | 3.61 | 3.75 |
| CSL44 | 3.20 | n.d. | 4.15 | 4.20 |
| CSL41 | 3.10 | n.d. | 3.75 | 3.16 |
| CSL42 | 3.23 | n.d. | 4.26 | 3.96 |
| CSL31 | 3.26 | n.d. | 4.01 | 4.07 |

n.d. = not determined at that time point

TABLE 10
C1-INH activity [IU/mL] of different C1-INH formulations at t₀ (start)
and after incubation at 35° C. at t₁ (10 days), t₂ (15 days), t₃ (21 days),
t₄ (31 days), t₅ (32 days), t₆ (60 days) and t₇ (90 days), t₈ (118
days), t₉ (139 days), t₁₀ (153 days) and t₁₁ (187 days)

| | at t₀ | at t₁ | at t₂ | at t₃ | at t₄ | at t₅ | at t₆ | at t₇ | at t₈ | at t₉ | at t₁₀ | at t₁₁ |
|---|---|---|---|---|---|---|---|---|---|---|---|---|
| CSL06 | 443 | n.d. | 405 | n.d. | n.d. | 362 | 349 | n.d. | n.d. | 197 | n.d. | n.d. |
| CSL17 | 426 | 418 | n.d. | 351 | 376 | n.d. | 270 | 205 | 173 | n.d. | 120 | 115 |
| CSL43 | 465 | n.d. | 447 | n.d. | n.d. | 411 | 378 | n.d. | n.d. | 231 | n.d. | n.d. |
| CSL44 | 477 | n.d. | 384 | n.d. | n.d. | 331 | 270 | n.d. | n.d. | 140 | n.d. | n.d. |
| CSL41 | 486 | n.d. | 472 | n.d. | n.d. | 428 | 387 | n.d. | n.d. | 212 | n.d. | n.d. |
| CSL42 | 484 | n.d. | 432 | n.d. | n.d. | 359 | 281 | n.d. | n.d. | 135 | n.d. | n.d. |
| CSL31 | 448 | n.d. | 396 | n.d. | n.d. | 328 | 265 | n.d. | n.d. | 132 | n.d. | n.d. |

n.d. = not determined at that time point

TABLE 11
HMWC levels [% of total protein] of different C1-INH
formulations by SEC HPLC at t₀ (start) and after incubation at 35° C. at
t₁ (15 days), t₂ (32 days), t₃ (90 days), t₄ (118 days), t₅ (139 days),
t₆ (153 days) and t₇ (188 days)

| | at t₀ | at t₁ | at t₂ | at t₃ | at t₄ | at t₅ | at t₆ | at t₇ |
|---|---|---|---|---|---|---|---|---|
| CSL06 | 2.40 | 6.69 | 10.27 | n.d. | n.d. | 22.55 | n.d. | n.d. |
| CSL17 | 2.26 | n.d. | n.d. | 25.02 | 28.3 | n.d. | 31.3 | 32.9 |
| CSL43 | 2.33 | 4.84 | 7.04 | n.d. | n.d. | 15.34 | n.d. | n.d. |
| CSL44 | 2.34 | 4.87 | 6.77 | n.d. | n.d. | 11.63 | n.d. | n.d. |
| CSL41 | 2.41 | 5.95 | 9.19 | n.d. | n.d. | 19.93 | n.d. | n.d. |
| CSL42 | 2.43 | 6.18 | 9.04 | n.d. | n.d. | 15.83 | n.d. | n.d. |
| CSL31 | 2.34 | 5.08 | 7.03 | n.d. | n.d. | 12.20 | n.d. | n.d. |

n.d. = not determined at that time point

TABLE 12
Fragment levels [% of total protein] of different C1-
INH formulations by SEC HPLC at t₀ (start) and after incubation at 35°
C. at t₁ (15 days), t₂ (32 days), t₃ (90 days), t₄ (118 days), t₅ (139 days),
t₆ (153 days) and t₇ (188 days)

| | at t₀ | at t₁ | at t₂ | at t₃ | at t₄ | at t₅ | at t₆ | at t₇ |
|---|---|---|---|---|---|---|---|---|
| CSL06 | 3.19 | 3.91 | 4.43 | n.d. | n.d. | 5.65 | n.d. | n.d. |
| CSL17 | 3.36 | n.d. | n.d. | 3.23 | 3.17 | n.d. | 3.31 | 3.50 |
| CSL43 | 3.19 | 4.19 | 4.11 | n.d. | n.d. | 4.96 | n.d. | n.d. |
| CSL44 | 3.20 | 5.13 | 4.13 | n.d. | n.d. | 5.71 | n.d. | n.d. |
| CSL41 | 3.12 | 4.28 | 4.09 | n.d. | n.d. | 4.76 | n.d. | n.d. |
| CSL42 | 3.23 | 4.72 | 4.68 | n.d. | n.d. | 5.41 | n.d. | n.d. |
| CSL31 | 3.26 | 3.88 | 3.93 | n.d. | n.d. | 5.71 | n.d. | n.d. |

n.d. = not determined at that time point

### Example 4: Determination of the Viscosity of C1-INH Formulations

A commercially available C1-INH product (Berinert®, 50 IU/mL after reconstitution), manufactured according to A. Feussner et al. (Transfusion 2014, 54: 2566-73), and a C1-INH concentrate, manufactured based on the Berinert® manufacturing process but concentrated to an increased C1-INH concentration (500 IU/mL after reconstitution), were used to prepare solutions of different C1-INH concentrations and different excipient concentrations (Table 13). Samples 1 and 2 contained the C1-INH and excipient concentrations as shown in Table 13. Sample 3 was prepared using buffer exchange by dialysis and contained the C1-INH and excipient concentrations as shown in Table 13. The C1-INH concentration was measured by a chromogenic assay (Berichrom C1-Inhibitor, Siemens).

TABLE 13
Excipient concentrations, pH and osmolarity
of three C1-INH formulations

| | Na₃-Citrat * | | | | | osmolarity |
|---|---|---|---|---|---|---|
| Sample | 2 H₂O | | Glycine | NaCl | pH | [mOsm/L] |
| 1 | 3.5 | g/L | 10.0 g/L | 8.5 g/L | 7.0 | 493 |
| 2 | 3.5 | g/L | 10.0 g/L | 8.5 g/L | 7.0 | 493 |
| 3 | 20 | mM | — | — | 6.9 | 80 |

The kinematic viscosity of the three samples was measured using a capillary viscometer according to Ubbelohde (Equipment no. 908829, capillary type 536 20/II, Schott-Geräte GmbH, Hofheim, Germany) based on DIN 51562, part 2, directly after preparation of the samples (t₀, initially) and after one week at +40° C. (t₁), see Table 14. To avoid any bioburden growth during storage at +40° C. the samples were protected by adjustment to 0.05% sodium azide.

Determination of the kinematic viscosity was done at +20° C. The capillary viscometer with a capillary diameter of 0.53 mm was tempered in a water bath. Samples were filled into the viscometer and incubated for 5 minutes to equilibrate to the measuring temperature. Performance of the measurements was done two times according to the instructions of the manufacturer of the viscometer and the means were calculated. Prior to the measurements, the capillary constant was verified using calibrated solutions from “Zentrum für Messen and Kalibrieren GmbH”, Bitterfeld, Germany. Note: To obtain the dynamic viscosity, the kinematic viscosity has to be multiplied with the mass density of the corresponding solutions which can be assumed to be about 1 g/cm³ (Dynamic Viscosity (cP)=Kinematic Viscosity (cSt)*Density (kg/m³)).

TABLE 14
Kinematic viscosity of three C1-INH formulations
comprising different buffer conditions at t₀ (start)
and after incubation at 40° C. at t₁ (1 week)

| | | kinematic | kinematic | Increase |
|---|---|---|---|---|
| | C1-INH at t₀ | viscosity at t₀ | viscosity at t₁ | of viscosity |
| Sample | [IU/mL] | [mm²/s] | [mm²/s] | [%] |
| 1 | 48 | 1.20 | 1.20 | 0% |
| 2 | 450 | 4.29 | 4.61 | 7% |
| 3 | 447 | 5.60 | 7.45 | 33% |

### Results

The sample 3 having sodium citrate as the only excipient shows a significantly higher viscosity compared to the formulations 1 and 2. The increase of viscosity of sample 2 (which is a formulation according to the invention) compared to the state of the art sample 1 is acceptable. The viscosity increase of sample 3 after one week at +40° C. was much higher than the increase of sample 2.

### Example 5: Pharmacokinetic Investigations Assessing the Bioequivalence of Berinert® and CSL06 Following Intravenous Administration in Rabbits

In this study, pharmacokinetic (PK) investigations were carried out to assess the bioequivalence of Berinert® and CSL06 of Example 1 to get an insight in potential changes of onset and modifications considering the pharmacokinetic shape of the plasma curve. Berinert® and CSL06 were administered each at a dose of 200 IU/kg via the intravenous route to 10 female Chinchilla Bastard (CHB) rabbits.

The area under the curve up to the last measured value after 96 hours (AUC_{0-96 h}) of human C1-INH:Ag levels in plasma was used as major bioequivalence parameter. Further PK parameters were investigated assessing bioequivalence: maximum plasma concentration (or, equivalently, in-vivo recovery), clearance, mean residence time, terminal half-life, volume of distribution at steady state, and terminal distribution volume. For the determination of PK parameters, plasma samples were drawn before administration and at 5 min, 30 min, 1 h, 4 h, 8 h, 24 h, 48 h, 72 h, and 96 h after administration and C1-INH:Ag plasma concentration was determined using an ELISA system.

The target variable for assessing bioequivalence of the two products was AUC_{0-96 h}, the area under the observed C1-INH:Ag plasma concentration time curve from administration until 96 h. The geometric mean values of AUC_{0-96 h} in the CSL06 group and in the Berinert® group were determined and the estimated geometric mean ratio was calculated to be 109% with a two-sided 90% confidence interval ranging from 100% to 118%. Therefore, average bioequivalence of the two products within the range of 80% to 125% was demonstrated for the variable AUC_{0-96 h} at a significance level of 5% (FIG. 4).

Average bioequivalence of CSL06 and Berinert® was likewise demonstrated for other pharmacokinetic parameters: Maximum plasma concentration (or equivalently, in-vivo recovery), clearance, mean residence time, terminal half-life, volume of distribution at steady state, and terminal distribution volume.

Geometric mean values of clearance were determined for CSL06 and Berinert® and the geometric mean ratio of clearance was calculated 92% with a 90% confidence interval from 85% to 100%.

Geometric mean values of terminal half-life were determined for CSL06 and Berinert® and the estimated geometric mean ratio of terminal half-life was calculated 101% with a 90% confidence interval from 94% to 109%.

Geometric mean values of terminal distribution volume for CSL06 and for Berinert® resulted in the estimated geometric mean ratio of terminal distribution volume of 93% with a 90% confidence interval from 84% to 102%.

In conclusion, the study supports the claim of average bioequivalence of CSL06 and Berinert® after single intravenous bolus infusion in rabbits.

### Example 6: Local Tolerance Study in New Zealand White Rabbits Following One Intravenous, Intra-Arterial, Subcutaneous or Intramuscular Injection of CSL06

In this study, the local tolerance of CSL06 (test item) following one intravenous, intra-arterial, subcutaneous or intramuscular injection in rabbits was evaluated. Four groups of two male and one female New Zealand White rabbits received the test item CSL06, once by intravenous or intra-arterial infusion, subcutaneous or intramuscular injection on the left side. CSL06 was administered once to New Zealand White Rabbits under a dosage-volume of 3 mL/injection by intravenous, intra-arterial infusion and subcutaneous injection and under a dosage-volume of 0.5 mL/injection by intramuscular injection. The right side of each animal was treated with 0.9% NaCl (control item) under the same experimental conditions. Following the administration, the animals were kept for a 4-day observation period. Each animal was checked daily for mortality or signs of morbidity and clinical signs. Cutaneous reactions were evaluated on day 1 before administration and 1 hour after treatment, on day 2, approximately 24 hours after the end of the administration and then once a day on days 3 and 4. Body weight was recorded once before group allocation, then on days 1 and 3. On completion of the observation period, animals were sacrificed and then subjected to a macroscopic post-mortem examination. A microscopic examination was performed on injection sites of all animals.

No unscheduled deaths and no systemic clinical signs were recorded. Body weight was unaffected by treatment with CSL06 independent of the route of administration. No CSL06-related local reactions were observed at intravenous, intra-arterial and intramuscular injection sites. Erythema and edema were observed at subcutaneous injection sites treated with CSL06 with a slightly higher incidence and/or severity when compared to control sites. No test item treatment-related macroscopic post-mortem changes were observed at the injection sites. At microscopy, occasional findings that were attributed to treatment were observed in intravenous (males), subcutaneous (females) and intramuscular (males and females) left injection sites, while no differences were observed with intra-arterial route. These lesions were within the normal range of reaction and consisted of minimal to slight perivenous collagen degradation and mononuclear cell infiltrate (intravenous route), collagen degradation (subcutaneous route) and mononuclear cell infiltrate (intramuscular route).

Under the experimental conditions of the study, a single intravenous or intra-arterial infusion or subcutaneous or intramuscular injection of CSL06 was clinically, locally and histologically well-tolerated in rabbits.

### Example 7: Local Tolerance Study in New Zealand White Rabbits Following One Subcutaneous Injection Comparing CSL06 and Berinert®

Three groups of two male and one female New Zealand White rabbits received either the test item, CSL06, the current marketed C1-inhibitor product Berinert® or placebo (Berinert® formulation buffer without the C1-INH), once by subcutaneous injection on the left side. The right side of each animal was treated with 0.9% NaCl (control item) under the same experimental conditions and acted as absolute control site. A constant dosage-volume of 3 mL/injection was used. Following the administration, the animals were kept for a 4-day observation period. Each animal was checked daily for mortality or signs of morbidity and clinical signs. Cutaneous reactions were evaluated on day 1 before administration and 1 hour after treatment, on day 2, approximately 24 hours after the end of the administration and then once a day on days 3 and 4. Body weight was recorded once before group allocation, then on days 1 and 3. On completion of the observation period, the animals were sacrificed and a macroscopic post-mortem examination was performed. A microscopic examination was performed on injection sites of all animals.

No unscheduled deaths and no clinical signs indicative of systemic toxicity were observed. Body weight was unaffected by the test item treatment. No reactions were observed at the sites injected with NaCl 0.9%. No significant differences were observed at the sites injected with CSL06, Berinert® or the placebo. Erythema was occasionally observed after treatment and sometimes persisted until day 3. The severity of this reaction was generally considered as slight. On day 4, two animals injected with one of the test items showed dryness at the injection sites. Hematoma was also seen at the sites injected with CSL06 or the placebo after treatment and lasted until day 4. No test item-related macroscopic post-mortem lesions were noted in any injection sites. Microscopy of the injection sites showed minimal acanthosis, occasionally associated with hyperkeratosis or minimal serocellular crust, and minimal or slight infiltrate of mononuclear inflammatory cells, associated with more or less heterophils, in the upper dermis. These minor changes observed in most animals regardless of the injection site (0.9% NaCl, placebo or test items) with similar incidence and severity were considered to be related to the administration procedure.

Under the experimental conditions of the study, CSL06, Berinert® and the placebo Berinert® formulation buffer were considered to be locally well tolerated after a single subcutaneous injection.

### Example 8: Local Tolerance Study in New Zealand White Rabbits Following One Subcutaneous Injection Comparing CSL43 and CSL43 Buffer without C1-INH

Three female New Zealand White rabbits received the test item, CSL43, once by subcutaneous injection to the dorsal right side of each animal. The dorsal left side of the same animals was treated with the liquid formulation buffer (CSL43 buffer without the C1-INH) under the same experimental conditions and acted as absolute control site. A constant dosage-volume of a 5 mL bolus was used for the injection. Following the administration, the animals were kept for a 72 h observation period. The animals were controlled visually to their physical condition and to clinical signs. Pictures of injection sites were taken at several time points and a score sheet was used. Blood parameters were determined using the ScilVet at baseline and 72 h. Skin biopsies were taken at necropsy and stored in 10% formalin solution for potential histological analyses.

No unscheduled deaths and no clinical signs indicative of systemic toxicity were observed. Immediately upon application of CSL43 or liquid formulation buffer and throughout the following observation period no signs of local reaction were macroscopically observed in any of the treated animals. At the end of the observation period of 72 h, necropsy was performed at the injection site. Here, in one animal a very slight and small haematoma (0.5 cm*1.0 cm) was observed in the subcutis below the injection site of CSL43 which was jugded to be procedure related. In all other animals, no local reactions or any other signs of treatment were observed. Additionally, upon analysis of the blood parameter, no treatment-related changes were observed in any of the animals.

Under the experimental conditions of the study, a single subcutaneous injection of the test item, CSL831, was clinically and locally well-tolerated in rabbits. No significant differences were observed at the sites injected with CSL43 or the liquid formulation buffer.

### Example 9

### Study Design

A phase II study was performed to characterize the pharmacokinetics, pharmacodynamics, and safety of CSL06 administered subcutaneously (SC) to 18 subjects with HAE.

Following a screening period of up to 30 days, subjects were sequentially allocated to one of six CSL06 treatment sequences by the treatment investigators (FIG. 5a). A single dose of Berinert 20 IU/kg was administered intravenously (IV) 2-7 days prior to the first CSL06 dosing period. Each subject received two of three possible CSL06 doses (1500 IU, 3000 IU, or 6000 IU administered as a short subcutaneous injection [500 IU/mL] twice-weekly) for two four-week treatment periods with a washout period of up to four weeks between periods. Administration of rescue medication (IV C1-INH) was permitted for breakthrough attacks.

### Study Population

Male or female subjects aged ≧18 with HAE with deficient C1-inhibitor (type I) or HAE with dysfunctional C1-inhibitor (type II), based on clinical history and confirmed by central laboratory testing at screening (C1-INH functional activity <50% or a C1-INH antigen level below the laboratory reference range), were eligible for the study. Subjects were required to have a body weight ≧50 and ≦110 kg at screening, and have experienced ≦5 HAE attacks within the 3 months prior to the screening visit, of which ≦1 occurred within 30 days prior to the screening visit.

Key exclusion criteria included current C1-INH prophylactic therapy, androgen therapy within 30 days of screening and any HAE-specific treatment within 7 days of screening.

### Endpoints

The primary endpoint was the mean trough C1-INH functional activity at the fourth week, based on modeling and simulation. Model-derived rather than observed trough levels were primarily chosen to account for the confounding nature of possible rescue IV C1-INH use during the study.

The secondary endpoints were the mean and mean change from baseline in trough C1-INH functional activity, C1-INH antigen level and C4 antigen levels at the fourth week of each dosing regimen, based on observed data.

Different summaries of treatment-emergent adverse events (AE) were conducted in order to assess safety.

### Dosing and Sample Collection

The dosing scheme and sample collection is illustrated in FIG. 5b. The initial single IV dose of C1-INH was administered to aid the pharmacokinetic (PK) model in accounting for any administration of IV C1-INH rescue doses, and to enable a within-study estimate of bioavailability of SC CSL06. C1-INH functional activity, C1-INH plasma concentrations and C4 antigen levels were assessed at several time points throughout each dosing period. Plasma C1-INH functional activity was additionally assessed immediately before CSL06 administration.

### PK and PD Measurements

Plasma C1-INH functional activity was assessed by a validated chromogenic assay (Berichrom C1-Inhibitor, Siemens; reference range: 70-130% of norm). Plasma C1-INH antigen (C1 reagent N-Antisera, Siemens Healthcare Diagnostics; reference range: 0.18-0.32 mg/L) and C4 antigen levels were assessed by nephelometry (C4 reagent, Beckman Coulter; reference range: 0.1-0.4 g/L). All measurements were performed at a central laboratory using a validated assay (CSL Behring GmbH, Marburg, Germany).

C4 antigen levels were defined as a pharmacodynamic (PD) parameter since C4 activity occurs downstream from C1-INH; C1-INH replacement would therefore affect C4 levels. Levels of C4 antigen have been shown to rise slowly over time following IV plasma-derived C1-INH (pdC1-INH) administration.

### PK and PD Analysis

The complete analysis set (18 patients who received ≧1 dose of CSL06 and provided ≧1 C1-INH functional measurement) was used for the primary endpoint analysis and to determine modeling-derived C1-INH functional activity. 12 subjects per dosing regimen were sufficient to provide an estimate of C1-INH activity, based on PK modeling of previous study results. The data for each treatment were summarized using descriptive statistics and a mixed model.

SC and IV C1-INH functional activity data were collectively subjected to a population-based approach using nonlinear mixed-effects modeling (NONMEM version 7.2). Exploratory PK characteristics of CSL06 were assessed by estimating typical and individual values for parameters such as clearance (CL) and volume of distribution (V) along with associated inter-individual variability. The influence of subject baseline characteristics was also investigated. Pharmacokinetic parameters such as CL, V, bioavailability (F), absorption rate constant (Kₐ), half-life (t_{1/2}), and incremental recovery were estimated with the final population PK model.

Pharmacokinetic simulations were conducted to examine whether steady-state trough levels of C1-INH functional activity were dependent on body weight. The body weight effect was evaluated by examining the distributions of model-predicted steady-state trough serum C1-INH functional activity at doses of 40 and 60 IU/kg and fixed doses of 3000 and 4500 IU, for baseline body weight ranges of <60 kg, 60-100 kg, and >100 kg. For the as-observed endpoint analysis the fourth week trough levels and increase in trough levels from baseline were summarized for C1-INH functional activity, C1-INH antigen levels and C4 antigen levels for each CSL06 dosing regimen using descriptive statistics.

### Safety Assessment

Safety and tolerability were evaluated by continuous observation of adverse events and by safety assessments that were conducted at specified times throughout the study. These assessments included infusion site tolerability, laboratory parameters, vital signs, body weight, physical examination, and concomitant medication usage. A risk assessment for deep vein thrombosis was also implemented based on earlier single case reports on side effects of thromboembolism in HAE patients using C1-INH concentrate.

### Results

### Study Subjects

22 subjects from eight study sites signed the informed consent. Of these, 18 patients were assigned to treatment by a computer generated list and received study drug. Four subjects signed the consent form but were not randomized, two because they did not meet the inclusion/exclusion criteria and two because the study had already reached the target of 18 subjects by the time they were found to be eligible. The demographics of the subjects are summarized in Table 15.

Pharmacokinetic Results after Subcutaneous CSL06

The mean as-observed steady-state trough C1-INH functional activity at the fourth week increased with CSL06 dose (FIG. 6); increase from baseline was 16.4%, 33.2% and 63.3% for the 1500 IU, 3000 IU and 6000 IU doses, respectively. C1-INH antigen levels also increased with CSL06 dose; increase from baseline in C1-INH antigen at the fourth week was 0.02 mg/mL, 0.05 mg/mL, and 0.14 mg/mL for the three dosing regimens, respectively.

### Population Pharmacokinetic Model

C1-INH functional activity was described by a linear 1-compartmental pharmacokinetic model with first-order absorption. The relationships between model parameters and the following baseline covariates were examined: age, gender, body weight, body mass index, ideal body weight, lean body mass, creatinine clearance, and C1-INH functional activity. The only statistically significant covariate effect on a model parameter identified was the effect of body weight on CL and V, which was described by the following relationships:

$${CL}_{i} = 0.398 \cdot {( \frac{{WT}_{i} ( kg )}{78.9 ( kg )} )}^{0.879} \cdot {exp}^{{η}_{CL}}$$

$${V}_{i} = 30.8 \cdot {( \frac{{WT}_{i} ( kg )}{78.9 ( kg )} )}^{0.669} \cdot {exp}^{{η}_{V}}$$

where CLᵢ is the individual value of clearance, Vᵢ the individual volume of distribution, and WTᵢ the body weight of subject i.

Goodness of fit plots of the model (FIG. 7) reveal that the model prediction was consistent with the observed data, as the points are uniformly distributed around the line of identity. Therefore, no systematic bias was evident.

### Model-Derived C1-INH Trough Levels and Pharmacokinetic Parameters

The primary endpoint was the mean trough C1-INH functional level at the fourth week, based on modeling and simulation (FIG. 6). A dose-dependent increase in mean C1-INH functional activity was observed, increasing from 14.6% at baseline to 31.7%, 44.3% and 80.5% for the 1500, 3000 and 6000 IU doses, respectively. The modeled steady-state trough C1-INH functional activity at the fourth week was similar to the as-observed C1-INH functional activity.

The model was also used to derive other exploratory PK parameters such as area under the activity-time curve from zero to end of dosing interval at steady state (AUC₍₀₋ₜ₎), maximum plasma C1-INH functional activity levels (Cₘₐₓ), average plasma activity at steady state (C_{avg}), incremental recovery, and elimination half-life. Table 16 summarizes the modeled PK parameters for functional C1-INH activity following IV and SC C1-INH administration. The bioavailability of SC CSL06 was 44%, with similar elimination half-life compared to IV C1-INH. Since the CSL06 doses used were not weight-based, body weight, body mass index and ideal body weight were assessed to determine their influence on median functional C1-INH levels. Not unexpectedly, a small negative relationship between body weight and steady-state trough levels was found for each dose regimen. Patients with a low body weight administered a fixed dose are predicted to achieve a relatively high functional C1-INH level compared to patients with a higher body weight. Therefore, a body weight-adjusted dosing is predicted to achieve the same level of activity across the range of body weights.

TABLE 15
Demographic and baseline characteristics (complete analysis set)

| | Dosing regimen | | | | | | | |
|---|---|---|---|---|---|---|---|---|
| | Berinert | | CSL06, 1500 IU | | CSL06, 3000 IU | | CSL06, 6000 IU | |
| Demographic characteristics | (N = 18) | | (N = 12) | | (N = 12) | | (N = 12) | |
| Age, years | | | | | | | | |
| Mean (SD) | 36.4 | (13.07) | 33.6 | (11.83) | 37.6 | (14.69) | 37.8 | (12.65) |
| Median (min, max) | 33.9 | (19, 69) | 30.3 | (19, 53) | 36.9 | (19, 69) | 35.2 | (24, 69) |
| Sex, male:female, n (%) | 7:11 | (38.9:61.1) | 4:8 | (33.3:67.7) | 5:7 | (41.7:58.3) | 5:7 | (41.7:58.3) |
| Weight, kg | | | | | | | | |
| Mean (SD) | 80.0 | (20.2) | 79.9 | (23.4) | 78.5 | (17.0) | 81.6 | (20.6) |
| Median (min, max) | 78.9 | (51.0, 110.0) | 71.2 | (51.0, 110.0) | 78.9 | (57.6, 106.5) | 83.3 | (51.0, 110.0) |
| Body mass index, kg/m² | | | | | | | | |
| Mean (SD) | 27.3 | (6.57) | 27.5 | (7.22) | 26.8 | (5.14) | 27.6 | (7.37) |
| Median (min, max) | 25.4 | (18.1, 40.9) | 25.4 | (19.1, 40.9) | 25.4 | (19.1, 34.5) | 27.5 | (18.1, 40.9) |
| HAE type, n (%) | | | | | | | | |
| Type I | 16 | (88.9) | 12 | (100) | 10 | (83.3) | 10 | (83.3) |
| Type II | 2 | (11.1) | 0 | (0) | 2 | (16.7) | 2 | (16.7) |
| Number of HAE attacks in | | | | | | | | |
| the preceding 3 months, n | | | | | | | | |
| Mean (SD) | 2.5 | (1.42) | 2.3 | (1.50) | 2.4 | (1.51) | 2.8 | (1.29) |
| Median (min, max) | 2.0 | (0, 5) | 2.0 | (0, 5) | 2.0 | (0, 5) | 2.0 | (1, 5) |
| Baseline as-observed C1-INH | | | | | | | | |
| functional activity, % | | | | | | | | |
| Mean (SD) | 14.6 | (8.02) | 15.4 | (8.02) | 11.3 | (7.10) | 17.1 | (8.05) |
| Median (min, max) | 15.2 | (1.3, 26.7) | 15.2 | (4.3, 26.7) | 9.9 | (1.3, 22.7) | 19.3 | (1.3, 26.7) |
| Baseline as-observed C1 | | | | | | | | |
| antigen level (mg/mL)* | | | | | | | | |
| n | 13 | | 8 | | 9 | | 9 | |
| Mean (SD) | 0.10 | (0.126) | 0.05 | (0.009) | 0.12 | (0.149) | 0.12 | (0.147) |
| Median (min, max) | 0.050 | (0.02, 0.44) | 0.047 | (0.03, 0.05) | 0.050 | (0.02, 0.44) | 0.053 | (0.02, 0.44) |
| Baseline as-observed C4 | | | | | | | | |
| antigen level (mg/dL)* | | | | | | | | |
| n | 17 | | 12 | | 11 | | 11 | |
| Mean (SD) | 8.6 | (9.51) | 6.6 | (3.10) | 9.2 | (11.7) | 9.9 | (11.7) |
| Median (min, max) | 7.0 | (2.2, 43.8) | 6.9 | (2.2, 10.8) | 7.8 | (2.2, 43.8) | 7.0 | (2.4, 43.8) |

*In some patients the values for baseline as-observed C1 and C4 antigen were below the limits of detection of 0.022 mg/mL and 1.67 mg/dL, respectively. Complete analysis set; 16 patients who rsceived ≧1 dose of CSL06 and provided ≧1 C1-INH functional measurement

TABLE 16
Modeled PK parameters for C1-INH activity

| | Berinert 20 IU/kg | CSL06, 1500 IU | CSL06, 3000 IU | CSL06, 6000 IU |
|---|---|---|---|---|
| | (N = 18) | (N = 12) | (N = 12) | (N = 12) |
| Cₘₐₓ, U/mL | 0.67 (0.562) | 0.38 (0.109) | 0.59 (0.154) | 1.09 (0.307) |
| AUC₍₀₋ₜ₎, U · h/mL | 38.9 (8.93) | 30.5 (11.31) | 45.3 (11.98) | 79.6 (25.36) |
| Cₐᵥ, U/mL | — | 0.36 (0.135) | 0.54 (0.142) | 0.95 (0.303) |
| Incremental recovery | 0.026 (0.0023) | 0.011 (0.0024) | 0.011 (0.0024) | 0.012 (0.0027) |
| ([U/mL]/[U/kg]) | | | | |
| t_{1/2} (h) | 52.8 (13.70) | 50.6 (12.35) | 51.5 (13.52) | 56.2 (15.02) |

| | |
|---|---|
| | Dose independent (n = 18) |
| CLss, L/h | 0.043 (0.015) |
| Vss, L | 3.06 (0.598) |
| Bioavailability | 0.44 |

Data are mean (SD).
AUC(0-t), area under the plasma concentration-time curve over a dosing interval; Cav, average C1-INH functional activity over a dosing interval; t½, elimination half-life; Cmax, maximum drug concentration in plasma; CLSS, ssteady-state clearance; VSS, steady-state volume of distribution.

### Pharmacodynamics

C4 antigen levels were also measured during the fourth week of each dose regimen. C4 antigen levels increased in a dose-dependent manner and normalized with all doses of CSL06. C4 levels of 11.1 mg/dL, 14.1 mg/dL and 18.4 mg/dL for the 1500 IU, 3000 IU and 6000 IU doses of CSL06, respectively were observed. C4 antigen levels increased from baseline at the fourth week by 4.3 mg/dL, 5.6 mg/dL and 9.1 mg/dL (normal level is 14 mg/dL) for the 1500 IU, 3000 IU and 6000 IU doses, respectively.

When the fixed dose of CSL06 was calculated as a dose per body weight, the mean C4 antigen level increased with the dose per body weight; the mean as-observed C4 antigen level at the fourth week was 11.3 mg/mL, 11.7 mg/mL, 18.0 mg/mL, and 18.2 mg/mL in the ≦20 IU/kg, >20 to ≦45 IU/kg, >45 to ≦90 IU/kg, and >90 IU/kg categories, respectively.

Modeled Twice-Per-Week CSL06 vs IV pdC1-INH Over 4 Weeks

The effect of dosing SC CSL06 vs IV 1000 IU pd C1-INH concentrate on C1-INH functional activity was examined in a post-hoc analysis by simulating three different SC regimens of 1500 IU, 3000 IU and 6000 IU twice-weekly for four weeks (FIG. 8). The simulated C1-INH functional activity time profiles showed a lower peak-to-trough ratio and more consistent exposures after SC administration compared to the current standard prophylactic treatment regimen of a twice-weekly injection of 1000 IU pd C1-INH concentrate.

### Safety

There were no CSL06-related serious adverse events (AEs) or deaths, or withdrawals due to adverse events throughout the study. No thromboembolic events were observed. Laboratory surveillance did not reveal any safety signals, and there was no evidence of inhibitory auto-antibody development.

The most common treatment-emergent adverse events (TEAEs) were local site reactions reported as pain, swelling, bruising and itching at the injection sites.

Most local site reactions were mild-to-moderate in intensity and resolved within 3 days. Moderate swellings at the injection site was reported in more subjects in the 6000 IU dose group (5/12 subjects) compared to the two other lower dose groups (2/12 in the 1500 IU and 1/12 in the 3000 IU dose group), which were likely related to the volume of injection. There was no apparent dose-relationship for other local site reactions.

HAE-related symptoms occurring during the study were reported as AEs. Overall, 29 HAE-related AEs were reported in seven patients during the course of the study. Of those 29 HAE events, 11 were reported during the four-week dosing intervals with SC CSL06. In the 1500 IU dose group, two out of 12 patients had two and five HAE attacks, respectively. In the 3000 IU dose group, two patients had one and three attacks, respectively. No patient in the 6000 IU dose group experienced any HAE symptoms during the four-week SC dosing period.

1. A stable pharmaceutical formulation comprising:
(a) C1-INH at a concentration of about 400 IU/mL-2,000 IU/mL; and
(b) sodium citrate having a calculated osmolarity of 20-120 mOsm/L or sodium di-hydrogen phosphate/di-sodium hydrogen phosphate having a calculated osmolarity of 60-120 mOsm/L; and
(c) one or more physiologically acceptable salt(s), other than the substances in (b), having a calculated osmolarity of 150-600 mOsm/L; or
one or more amino acid(s) selected from glycine and/or one or more basic L-amino acid(s) and/or one or more acidic L-amino acid(s) or a salt/salts thereof having a calculated osmolarity of 50-500 mOsm/L; or
one or more physiologically acceptable salt(s), other than the substances in (b), and one or more amino acid(s) selected from glycine and/or one or more basic L-amino acid(s) and/or one or more acidic L-amino acid(s) or a salt/salts thereof having together a calculated osmolarity of 80-740 mOsm/L,
wherein the overall calculated osmolarity of the formulation is 170-800 mOsm/L.

2. The pharmaceutical formulation of claim 1, wherein the physiologically acceptable salt is a physiologically acceptable sodium salt.

3. The pharmaceutical formulation of claim 1, wherein the basic L-amino acid is arginine, lysine, and/or histidine or a salt/salts thereof.

4. The pharmaceutical formulation of claim 1, wherein the acidic L-amino acid is glutamic acid and/or aspartic acid or a salt/salts thereof.

5. The pharmaceutical formulation of claim 1, wherein the pH of the formulation is between about 6.7 and about 7.5.

6. The pharmaceutical formulation of claim 1, wherein the formulation comprises:
(a) about 400-625 IU/mL C1-INH;
(b) about 20-120 mOsm/L sodium citrate;
(c) about 50-300 mOsm/L glycine; and
(d) about 190-400 mOsm/L sodium chloride,
wherein the overall calculated osmolarity of the formulation is 260-800 mOsm/L.

7. The pharmaceutical formulation of claim 1, wherein the melting temperature of C1-INH, measured by DSF, in said formulation is about 55° C. or higher.

8. The pharmaceutical formulation of claim 1, wherein the formulation further comprises:
(a) a detergent selected from the group consisting of PS80 (polysorbate 80) and PS20 (polysorbate 20); and/or
(b) a preservative and/or antioxidant selected from the group consisting of benzylalcohol, cresol, phenol, methionine and glutathione.

9. The pharmaceutical formulation of claim 1, wherein the C1-INH is human C1-INH.

10. The pharmaceutical formulation of claim 1, wherein the formulation comprises an absolute amount of C1-INH of at least 1,200 IU, at least 1,500 IU, or at least 1,800 IU per finished dosage form.

11. The pharmaceutical formulation of claim 1, wherein the formulation is
(a) obtainable by reconstitution of a lyophilized powder with a suitable liquid, or
(b) provided as a liquid formulation.

12. The pharmaceutical formulation of claim 1, wherein the formulation can be administered via subcutaneous administration or via intravenous administration.

13. The pharmaceutical formulation of claim 1, wherein a patient can self-administer the formulation.

14. The pharmaceutical formulation of claim 1, wherein the kinematic viscosity of the formulation at +20° C. is below 10 mm²/s, below 8 mm²/s, below 6 mm²/s, or below 5 mm²/s.

15. The pharmaceutical formulation of claim 1, wherein the formulation comprises less than 10% of high molecular weight components (HMWC), less than 8% of HMWC, less than 5% of HMWC, or less than 3% of HMWC.

16. A method of treating a disorder related to kinin formation; a disorder related to an ischemia-reperfusion injury (IRI); retinopathy; or in preventing rejection of transplanted tissue in a patient, comprising administering to a patient in need thereof the pharmaceutical formulation of claim 1.

17. A kit comprising the pharmaceutical formulation of claim 1 as a lyophilized powder and a respective volume of a suitable liquid for reconstitution.

18. A kit comprising the pharmaceutical formulation of claim 1 and at least one syringe and/or one needle.

19. A syringe prefilled with a liquid pharmaceutical formulation of claim 1.

20. The pharmaceutical formulation of claim 2, wherein the physiologically acceptable salt is selected from sodium chloride, di-sodium EDTA, sodium acetate, sodium succinate, and sodium sulphate.

21. The pharmaceutical formulation of claim 7, wherein the melting temperature of C1-INH, measured by DSF, in said formulation is about 55-60° C.

22. The pharmaceutical formulation of claim 9, wherein the C1-INH is derived from human plasma.

23. The method of claim 16, wherein the disorder related to kinin formation is hereditary angioedema (HAE), HAE type I, HAE type II, or HAE type III.

24. The method of claim 16, wherein the disorder related to an ischemia-reperfusion injury is due to surgical intervention.

25. The method of claim 24, wherein the surgical intervention is vascular surgery, cardiac surgery, neurosurgery, trauma surgery, cancer surgery, orthopedic surgery, transplantation, minimally invasive surgery, or insertion of a device for delivery of a pharmacologically active substance or for mechanical removal of complete or partial obstructions.

26. The method of claim 16, wherein the treating of the disorder is an acute and/or prophylactic treatment.
